(19) United States
(12) Patent Application Publication
(10) Pub. No.: US 2026/0034227 A1
(43) Pub. Date: Feb. 5, 2026

(54) AZLACTONE-BASED POLYMERS AS A SCAFFOLD FOR DIVERSE APPLICATIONS IN DRUG AND GENE DELIVERY

(71) Applicant: University of Mississippi, University, MS (US)

(72) Inventors: Adam Eugene Smith, University, MS (US); Thomas Anthony Werfel, Oxford, MS (US); Sk Arif Mohammad, Oxford, MS (US); Alexander Webb Fortenberry, University, MS (US)

(21) Appl. No.: 18/997,202

(22) PCT Filed: Jul. 18, 2023
(86) PCT No.: PCT/US2023/070360

§ 371 Date: Jan. 21, 2025

Related U.S. Application Data
(60) Provisional application No. 63/368,718, filed on Jul. 18, 2022.

(51) Int. Cl. A61K 47/58; A61K 31/352; A61K 31/37; A61K 31/4745; A61K 31/7105; A61K 31/711; A61K 38/16; C08F 126/06

(52) U.S. Cl. CPC A61K 47/58; A61K 31/352; A61K 31/37; A61K 31/4745; A61K 31/7105; A61K 31/711; A61K 38/16; C08F 126/06

(57) ABSTRACT

In one aspect, the disclosure relates to charge-shifting polymers including a polymeric backbone, one or more tertiary amine-containing pendant groups, and at least one linker between the polymeric backbone and each of the one or more pendant groups, wherein the at least one linker comprises a thioester. Also disclosed are methods of making the same, compositions comprising the charge-shifting polymers and at least one therapeutic molecule, and methods of controlled intracellular release of therapeutic molecules including, but not limited to, small molecules, peptides and proteins, and nucleic acids using the same. This abstract is intended as a scanning tool for purposes of searching in the particular art and is not intended to be limiting of the present disclosure.

### CROSS-REFERENCE TO RELATED APPLICATIONS

This application claims the benefit of U.S. Provisional Application No. 63/368,718, filed on Jul. 18, 2022, which is incorporated herein by reference in its entirety.

### STATEMENT REGARDING FEDERALLY SPONSORED RESEARCH OR DEVELOPMENT

This invention was made with government support under grant number P20 GM130460-01A1 7937, awarded by the National Institutes of Health, and NSF CAREER 2141666, awarded by the National Science Foundation. The government has certain rights in the invention.

### BACKGROUND

Charge-shifting polymers are a class of polyelectrolytes that can gradually change net charge in response to stimuli such as changes in pH or glutathione concentration. While examples include anionic-to-cationic and cationic-to-zwitterionic shifts, polymers exhibiting cationic-to-anionic shifts are the most widely employed. Charge-shifting polycations can form a strong inter-polyelectrolyte complex with polyanions which will dissociate upon a reduction in the net cationic charge of the polymer. This property makes charge-shifting polycations intriguing for several biomedical applications, including DNA delivery, drug delivery, and protein delivery.

A common example of a charge-shifting polycation is poly(dimethylamino)ethyl acrylate (PDMAEA), which has side chains that possess tertiary amine moieties connected to the polymer via an ester bond. Since esters can undergo base-catalyzed hydrolytic degradation to form negatively charged carboxylic acids, PDMAEA can undergo a cationic-to-anionic net charge transition after cleavage of the tertiary amine via hydrolysis of the ester bond.

2-vinyl-4,4-dimethylazlactone (VDMA) has garnered attention for the synthesis of charge-shifting polymers as the azlactone moiety can undergo ring-opening reactions when exposed to a variety of nucleophilic species, including primary amines, hydroxyls, and thiols. VDMA can be polymerized utilizing controlled polymerization techniques like reversible addition-fragmentation chain transfer (RAFT) polymerization allowing the facile synthesis of a “reactive platform” for post-polymerization modifications to afford a broad range of polymers derived from the same parent polymer, thus having the same degree of polymerization while retaining the azlactone functionality. For example, PVDMA can be reacted with primary amines to prepare: thin films with superhydrophobic properties; protein-polymer conjugates; and thermo-responsive materials. Interestingly, when PVDMA is reacted with hydroxyl groups, it forms an ester bond that is susceptible to hydrolytic degradation. If the hydroxyl moiety contains a tertiary amine, then upon hydrolysis, the tertiary amine-functionalized PVDMA changes net charge (owing to the resulting carboxylic acid groups on the hydrolyzed PVDMA).

Depending on the pH in which this charge transition occurs, the polymer could transition from cationic-to-anionic, neutral-to-anionic, or cationic-to-neutral. This opens the door to applications such as the delivery of biomolecules where anionic species can be released from positively-charged PVDMA-based polymers upon hydrolysis and subsequent cationic-to-anionic transition. For example, PVDMA functionalized with tertiary amine side chains via ester linkages has been used to prepare multilayered films that can release plasmid DNA (pDNA) upon hydrolysis of the side chains. In a subsequent study, pDNA-containing ultrathin films based on PVDMA functionalized with tertiary amine side chains connected to the backbone via amide and ester linkages was prepared. The rate of hydrolysis (and release of pDNA) could be controlled for up to three months under physiological conditions. In another notable study, the rate of hydrolytic degradation of PVDMA functionalized with tertiary amine-functionalized alcohols was investigated, where the length of the alkyl spacer between the amine and resulting ester bond is either two or three carbons. Under physiological conditions (pH 7.4 and 37° C.), it was reported that the half-life was six days for polymers with a two-carbon spacer. In contrast, the half-life was 200 days for a three-carbon spacer, allowing the hydrolytic degradation to be tuned by manipulating the ratio of these moieties in PVDMA. It was hypothesized that the reasons for differing degradation rates arise from a combination of two influences: differences in the hydrophobicity of the side chains and the mechanisms for the ester hydrolysis with the potential of intramolecularly-assisted hydrolysis more likely for the polymer with the shorter alkyl spacer.

The hydrolytic degradation of a structurally-similar charge shifting polymer, PDMAEA, with an alkyl spacer of two carbons between the tertiary amine and ester bond at various pHs and temperatures has also been investigated. It was proposed that the amine group can assist hydrolysis of the ester group via a 5-membered or 7-membered intramolecular mechanism under acidic and basic conditions, respectively.

While the hydrolysis of esters can be slow depending on pH and temperature, the rate of hydrolysis of thioesters can be orders of magnitude higher depending on pH, temperature, and sterics around the thioester bond. Additionally, thioesters are susceptible to degradation via exposure to glutathione (GSH), which can accelerate the degradation under physiologically-relevant conditions and can broaden the “kinetic window,” making thioesters particularly intriguing for biological applications such as intracellular delivery. Therefore, the functionalization of PVDMA with thiols could result in materials that undergo hydrolytic degradation significantly faster than the corresponding esters, which could open the door to applications requiring a faster rate of polymer hydrolysis and charge-shifting.

Meanwhile, most small molecule drugs (i.e., pharmaceutical agents) show poor bioavailability and insufficient pharmacokinetics because of their physicochemical characteristics, such as insolubility, or lack of in vivo stability. The bioavailability of small molecule drugs depends on molecular flexibility, the polar surface area, and the total number of hydrogen bonds, which are often indirectly associated with the drug's molecular weight. Polymeric drug delivery systems can improve the efficacy of these agents by making these drugs more water-soluble, extending their circulation time, enhancing the drug's localization in the target tissue, and limiting their exposure to healthy tissues. Recently, the Chilkoti group reported drug-loaded nanoparticle synthesis via ring-opening polymerization (ROP) composed of a cyclic polymerizable group attached to pendant drug groups through a cleavable linker. They used poly(ethylene glycol) methyl ether (mPEG) as a macroinitiator for the synthesis of diblock copolymers of mPEG and drugamers (or drug-based monomers) that self-assemble into spherical micelles with a PEG corona. The same group reported paclitaxel-loaded drug nanoparticles via ROP, and others have employed ROP for synthesizing biodegradable polypeptides, polyesters, polyphosphoesters, and polycarbonates. However, the direct polymerization of pharmaceutically active monomers is limited by the choice of available drug-based monomers, harsh synthesis conditions, and batch-to-batch variation in terms of polymer molecular weight and polydispersity.

Alternatively, platforms that allow large-scale synthesis of a single batch of polymer followed by post-polymerization modification would be highly desirable for ensuring consistency between batches and broadening the number of drugs that can be incorporated into macromolecular drug design.

Despite advances in PVDMA functionalization research, there is still a scarcity of strategies for controlling side-chain hydrolysis rates for biomedical applications including DNA delivery, protein delivery, and drug delivery. The hydrolytic degradation of PVDMA functionalized with a library of tertiary amine-containing side chains, while potentially useful for tunability of DNA, protein, and drug delivery, is not yet fully understood. Furthermore, it is desirable to more fully characterize the effects of side chains connected to the polymer backbone via amide, ester, or thioester linkages to examine the effect of temperature and pH on the rate of degradation of these groups to provide a greater understanding of the kinetics of hydrolysis of the PVDMA-based charge shifting polycations as well as bioavailability of the cargo molecules of these charge-shifting polymers. These needs and other needs are satisfied by the present disclosure.

### SUMMARY

In accordance with the purpose(s) of the present disclosure, as embodied and broadly described herein, the disclosure, in one aspect, relates to charge-shifting polymers including a polymeric backbone, one or more tertiary amine-containing pendant groups, and at least one linker between the polymeric backbone and each of the one or more pendant groups, wherein the at least one linker comprises a thioester. Also disclosed are methods of making the same, compositions comprising the charge-shifting polymers and at least one therapeutic molecule, and methods of controlled intracellular release of therapeutic molecules including, but not limited to, small molecules, peptides and proteins, and nucleic acids using the same.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows GPC elution time trace of the PVDMA scaffold. Mₙ=22,600 g/mol, Ð=1.03.

FIG. 2 shows functionalization of PVDMA with primary-amine, hydroxyl, and thiol functional tertiary amines followed by the hydrolysis of the modified PVDMA.

FIG. 3 shows FTIR spectra of PVDMA, amide-modified PVDMA, ester-modified PVDMA, and thioester-modified PVDMA. The characteristic carbonyl stretching band associated with lactone at 1810 cm⁻¹ is not present in the modified samples indicating complete conversion.

FIG. 4 shows the kinetics of the hydrolysis of ester-modified and amide-modified PVDMA at 25° C. and 50° C. and PH 5.5, pH 7.5, and pH 8.5. The rate of hydrolysis of the ester-modified PVDMA increased with both temperature and pH, while no noticeable degradation of the amide-modified PVDMA was observed during the periods of the experiments.

FIGS. 5A-5B show hydrolysis of the thioester-modified PVDMA and subsequent thiol-thiol coupling of the small-molecule disulfide byproduct (FIG. 5A) and ¹H NMR spectra monitoring the hydrolysis of the thioester-modified PVDMA at 50° C. for different times and pH values (FIG. 5B). The shaded regions of the spectra correspond to the protons labeled with the same color in FIG. 5A. As hydrolysis proceeds, peaks associated with the small-molecule thiol product appear along with peaks associated with the disulfide byproduct formed from the small-molecule thiols.

FIG. 6 shows agarose gel experimental results of polyplexes formulated from tertiary amine-functionalized PVDMA with thioester linkages. The left-most cell is free DNA formulated with no polymers. The rest of the cells are polyplexes formulated with N/P ratios of approximately 20/1 with varying equivalents of glutathione (GSH) concentrations relative to the number of tertiary amine moieties.

FIG. 7 shows synthesis of tertiary amine-, camptothecin- and doxorubicin-modified PVDMA.

FIG. 8A shows a basic schematic showing the complexation of nucleic acids via cationic tertiary amine-modified PVDMA. FIG. 8B shows the complexation of DNA with tertiary amine-modified PVDMA at various N/P ratios. FIG. 8C shows the hydrolysis-induced release of DNA from the polyplexes at various time points. FIG. 8D shows a schematic demonstrating the release of coumarin moieties via hydrolysis. FIG. 8E shows the release of coumarin from PVDMA vs. time at pH 5.5 and pH 7.4.

FIGS. 9A-9F show biocompatibility and cell uptake of DMAE-modified 3-bromoacetyl coumarin (DBAC)-PVDMA polymers. FIG. 9A shows cell viability of Hela cells after incubation with varying concentrations of DBAC-modified PVDMA for 24 h (Data represent mean±s.d., n=4), FIG. 9B shows cell viability of HEK-293 cells after incubation with different concentrations of DBAC-modified PVDMA for 24 h (Data represent mean±s.d., n=4), FIG. 9C shows a schematic synthesis diagram of fluorescently-labeled PVDMA polymer (DBAC-TMR-PVDMA), confocal microscopy images of DBAC-TMR-PVDMA in HEK-293 cells (FIG. 9D) upon 10 min treatment, (FIG. 9E) upon overnight incubation, and (FIG. 9F) time-lapse following 10 min treatment.

FIGS. 10A-10D show subcellular colocalization of DBAC and TMR-PVDMA polymer. (FIG. 10A) Fluorescence imaging showing the distribution of DBAC (C), TMR (R), and LysoTracker Green (L) dyes in HEK-293 cells. (FIG. 10B) Fluorescence imaging showing the overlap of all 3 dyes in HEK-293 cells. DBAC, TMR, and LysoTracker Green dyes, (FIG. 10C) Fluorescence imaging showing the overlap between 2 dyes in HEK-293 cells. Overlap of DBAC and TMR (CR), DBAC and LysoTracker Green (CL), TMR and LysoTracker Green (RL) dyes, respectively. (FIG. 10D) Pearson's correlation graph showing the colocalization relationship between 2 dyes (left-to-right: CR, CL, and RL).

FIGS. 11A-11F show IV Injection of DBAC-TMR-PVDMA extends DBAC circulation and drives kidney accumulation. Pharmacokinetics of DBAC-TMR-PVDMA polymers in (FIG. 11A) plasma, (FIG. 11B) lungs, (FIG. 11C) heart, (FIG. 11D) spleen, (FIG. 11E) liver, and (FIG. 11F) kidney after i.v. injection. (Data represent mean±s.d., n=3 at each time point.)

FIGS. 12A-12F show oral administration of DBAC-TMR-PVDMA drives high accumulation in the GI tract with limited systemic absorption. Pharmacokinetics of DBAC-TMR-PVDMA polymers in the (FIG. 12A) GI tract, (FIG. 12B) plasma, (FIG. 12C) lungs, (FIG. 12D) kidney, (FIG. 12E) liver, and (FIG. 12F) spleen after oral administration. (Data represent mean±s.d., n=3 at each time point.)

FIGS. 13A-13D show post-polymerization modification of PVDMA with DBAC and doxorubicin. (FIG. 13A) ¹H-NMR spectra of DBAC-modified PVDMA polymer in MeOD, (*) solvent peak, (FIG. 13B) FT-IR spectrum of homopolymer PVDMA, DBAC-modified PVDMA, (FIG. 13C) ¹H-NMR spectra of doxorubicin-modified PVDMA polymer in DMSO-d6, (*) solvent peak, (FIG. 13D) FT-IR spectrum of homopolymer PVDMA, free doxorubicin, and doxorubicin-modified PVDMA polymer.

FIG. 14A shows ¹H NMR spectrum of PVDMA homopolymer in CDCl₃, (*) solvent peak, FIG. 14B shows an FTIR spectrum of PVDMA. FIGS. 14C-14E show GPC elugrams of various PVDMA homopolymers.

FIG. 15 shows a ¹H NMR spectrum of DBAC in D₂O, (*) solvent peak.

FIG. 16 shows an FTIR spectrum of DBAC.

FIG. 17 shows a ¹H-NMR spectrum of camptothecin-modified PVDMA polymer in DMSO-d₆, (*) solvent peak.

FIG. 18 shows an FTIR spectrum of PVDMA homopolymer and camptothecin-modified PVDMA polymer.

FIG. 19 shows a ¹H NMR spectrum of functionalization of PVDMA with tertiary amine via ester linkages in D₂O, (*) solvent peak.

FIG. 20A shows a plot of absorbance of DBAC at pH 5.5 with respect to 316 nm vs various ranges of DBAC concentration in mg/mL, FIG. 20B shows a plot of absorbance of DBAC at pH 7.4 with respect to 316 nm vs various ranges of DBAC concentration in mg/mL, FIG. 20C shows absorbance of DBAC release with a period of time from dialysis bag at pH 5.5, and FIG. 20D shows absorbance of DBAC release with period of time from dialysis bag at pH 7.4.

FIG. 21 shows a ¹H NMR spectrum PVDMA polymer functionalized with both DBAC and tetramethyl rhodamine cadaverine in DMSO-d₆, (*) solvent peak.

FIG. 22 shows an FTIR spectrum of PVDMA polymer functionalized with both DBAC and tetramethyl rhodamine cadaverine.

FIG. 23 shows a ¹H-NMR spectrum of DBAC-modified PVDMA polymer in DMSO-d₆, (*) solvent peak.

FIG. 24 shows a ¹H-NMR spectrum in D₂O of DBAC-modified PVDMA polymer after hydrolysis at pH 7.4, (*) solvent peak.

FIG. 25 shows a plot of fluorescence intensity vs. concentration of fluorescently labeled PVDMA polymer.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Poly 2-vinyl-4,4-dimethylazlactone (PVDMA) has received much attention as a “reactive platform” to prepare charge-shifting polycations via post-polymerization modification with tertiary amines that possess primary amine or hydroxyl reactive handles. Upon hydrolysis of the resulting amide or ester linkages, the polymers can undergo a gradual transition in net charge from cationic to anionic. Herein, a systematic investigation of the hydrolysis rate of PVDMA-derived charge-shifting polymers is described. PVDMA was modified with tertiary amines bearing either primary amine, hydroxyl, or thiol reactive handles. The resulting polymers possessed tertiary amine side chains connected to the backbone via amide, ester, or thioester linkages. The hydrolysis rates of each PVDMA derivative were monitored at 25° C. and 50° C. at pH values of 5.5, 7.5, and 8.5. While the hydrolysis rate of the amide-functionalized PVDMA was negligible over the period investigated, the hydrolysis rates of the ester- and thioester-functionalized PVDMA increased with increasing temperature and pH. Interestingly, the hydrolysis rate of the thioester-functionalized PVDMA appears to be more rapid than the ester-functionalized PVDMA at all pH values and temperatures investigated. These results can be utilized to inform the future preparation of PVDMA-based charge-shifting polymers for biomedical applications.

Further disclosed are the cell trafficking, and in vivo pharmacokinetics of PVDMA-based stimuli-responsive materials. In one aspect, reversible addition-fragmentation chain-transfer (RAFT) polymerization can be used to synthesize well-defined, narrowly-dispersed PVDMA (Scheme 1a). In another aspect, this strategy works for a post-polymerization modification with a variety of structurally distinct drugs. In a still further aspect, using this methodology, more than one drug can be attached to the polymer scaffold. In another aspect, the drug loading and release can be tuned by adjusting the ratio of the azlactone ring to the drug and creating linkages broken down by relevant in vivo triggers, such as pH and redox. In one aspect, PVDMA scaffolds can be used in the disclosed methods to produce representative delivery vehicles via post-polymerization modification (Scheme 1). In a further aspect, PVDMA modified with tertiary amine-containing DMAE has been shown to form reversible complexes with nucleic acids (Scheme 1b-c). Also disclosed herein is a strategy by which coumarin-based drugs can modify PVDMA following an alkylation reaction of the tertiary amine of DMAE with 3-bromoacetyl coumarin (DBAC; Scheme 1d). In another aspect, PVDMA can be modified with the chemotherapeutic drugs doxorubicin (Scheme 1e) and camptothecin (Scheme 1f) to demonstrate the broad applicability of this approach. In an aspect, the potential of the formulated polymer-drug conjugates was determined by determining the in vitro release kinetics, cellular uptake, and cytotoxicity in tumor and kidney cell lines. Also disclosed are the in vivo pharmacokinetics and biodistribution of DBAC-modified PVDMA polymers in mice, representing the evaluation of PVDMA-based polymers in vivo for the first time.

In one aspect, disclosed herein is a charge-shifting polymer including a polymeric backbone, one or more tertiary amine-containing pendant groups, and at least one linker between the polymeric backbone and each of the one or more pendant groups, wherein the at least one linker can be or include a hydrolyzable group. In some aspects, the at least one linker can further be or include an amide, ester, thioester, or any combination thereof.

In one aspect, the charge-shifting polymer has a number-average molecular weight (Mₙ) of from about 10,000 Da to about 24,000 Da, or from about 10,000 Da to about 20,000 Da, about 10,000 Da to about 15,000 Da, about 15,000 Da to about 20,000 Da, or about 22,600 Da. In another aspect, the charge-shifting polymer has a polydispersity of from about 1.00 to about 1.05, about 1.01 to about 1.03, or of about 1.03.

In one aspect, the charge-shifting polymer has a hydrolytic half-life of from about 15 hours to about 1800 hours, or about 15, 50, 100, 150, 200, 250, 300, 350, 400, 450, 500, 600, 700, 800, 900, 1000, 1100, 1200, 1300, 1400, 1500, 1600, 1700, or about 1800 hours, or a combination of any of the foregoing values, or a range encompassing any of the foregoing values. In one aspect, the hydrolytic half-life can be measured at a temperature of from about 25° C. to about 50° C., or at about 25, 30, 35, 40, 45, or about 50° C., or a combination of any of the foregoing values, or a range encompassing any of the foregoing values. In still another aspect, the hydrolytic half-life can be measured at a pH of from about 5.5 to about 8.5, or at about 5.5, 6, 6.5, 7, 7.5, 8, or about 8.5, or a combination of any of the foregoing values, or a range encompassing any of the foregoing values.

In any of these aspects, the charge-shifting polymer is biocompatible. In one aspect, disclosed herein is a composition that includes the disclosed charge-shifting polymers and at least one therapeutic molecule. In another aspect, the at least one therapeutic molecule can be a small molecule, a peptide, a protein, DNA, RNA, or any combination thereof.

In a further aspect, the small molecule can be selected from coumarin, a coumarin derivative, camptothecin, doxorubicin, tetramethylrhodamine cadaverine, or any combination thereof. In a further aspect, the coumarin derivative can be DMAE-modified 3-bromoacetyl coumarin (DBAC). In some aspects, the at least one therapeutic molecule can be two small molecules such as, for example, DBAC and tetramethylrhodamine cadaverine. Further in this aspect, the DBAC and tetramethylrhodamine cadaverine can be present in a ratio of from about 99:1 to about 99.5:0.5.

In an aspect, the charge-shifting polymer can include at least a first hydrolyzable group and a second hydrolyzable group, wherein the first and second hydrolyzable groups have different hydrolytic half-lives under a single set of pH and temperature conditions. In a further aspect, the first hydrolyzable group can have a hydrolytic half-life that is shorter than a half-life of the second hydrolyzable group, or longer than a half-life of the second hydrolyzable group. In some aspects, the charge-shifting polymer further includes a third hydrolyzable group having a different hydrolytic half-life from both the first hydrolyzable group and the second hydrolyzable group under a single set of pH and temperature conditions.

Also disclosed herein is a method for controlled delivery of at least one therapeutic molecule to a subject, the method including administering the disclosed composition to the subject. In a further aspect, in the disclosed method, hydrolysis of the charge-shifting polymer causes the at least one therapeutic molecule to disassociate from the charge-shifting polymer. In an aspect, hydrolysis of the charge-shifting polymer occurs intracellularly. In another aspect, hydrolysis of the charge-shifting polymer occurs in lysosomes.

In one aspect, the hydrolyzable group can be a thioester and the at least one therapeutic molecule can dissociate from the charge-shifting polymer after uptake by a cell, wherein the cell includes free glutathione. In a further aspect, the free glutathione in the cell can have a concentration of from about 1 nm to about 10 nm, or from about 1 nm to about 5 nm, or from about 5 nm to about 10 nm.

In any of these aspects, the composition can be administered orally or intravenously. In a further aspect, the composition can be administered orally and the charge shifting polymer has a terminal half life of from about 5 hours to about 9 hours, from about 5 hours to about 6 hours, from about 6 hours to about 8 hours, or from about 7 hours to about 9 hours.

In a further aspect, the subject can be a mammal such as, for example, a human, mouse, rat, hamster, rabbit, guinea pig, cat, dog, sheep, goat, swine, cattle, or horse.

In another aspect, disclosed herein is a method for making a charge-shifting polymer, the method including at least the step of contacting an azlactone polymer with a tertiary amine, wherein the tertiary amine has a structure of Formula I:

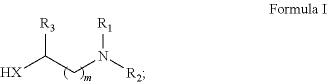

wherein m is from 0 to 10;
wherein X is selected from S, O, NH, or any combination thereof;
wherein R₁ and R₂ are independently selected from a linear or branched C1-C10 alkyl group or wherein R₁, N, and R₂ together form a 3- to 6-membered cycloalkyl or heterocycloalkyl group;

and wherein R₃ is hydrogen or a linear or branched C1-C10 alkyl group.

In another aspect, the compound of Formula I can be:

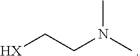

In another aspect, in the disclosed method the azlactone polymer and tertiary amine can further be contacted with a base such as, for example 1,8-diazabicyclo(5.4.0)undec-7-ene (DBU).

In one aspect, the azlactone polymer can be poly 2-vinyl-4,4-dimethylazlactone (PVDMA). In another aspect, the method can be conducted at about 45° C. for from about 24 hours to about 72 hours, or for about 24, 30, 36, 42, 48, 54, 60, 66, or about 72 hours, or a combination of any of the foregoing values, or a range encompassing any of the foregoing values. Also disclosed herein are charge-shifting polymers produced by the disclosed methods.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by,” “comprising,” “comprises,” “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a linker,” “a side chain,” or “a hydrolysis rate,” include, but are not limited to, mixtures, combinations, or groupings of two or more such linkers, side chains, or hydrolysis rates, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y.’ The range can also be expressed as an upper limit, e.g. ‘about x, y, z, or less’ and should be interpreted to include the specific ranges of ‘about x,’ ‘about y’, and ‘about z’ as well as the ranges of ‘less than x’, less than y′, and ‘less than z’. Likewise, the phrase ‘about x, y, z, or greater’ should be interpreted to include the specific ranges of ‘about x,’ ‘about y,’ and ‘about z’ as well as the ranges of ‘greater than x,’ greater than y,’ and ‘greater than z.’ In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

Unless otherwise specified, pressures referred to herein are based on atmospheric pressure (i.e. one atmosphere).

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### ASPECTS

The present disclosure can be described in accordance with the following numbered Aspects, which should not be confused with the claims.

Aspect 1. A charge-shifting polymer comprising:
(a) a polymeric backbone;
(b) one or more tertiary amine-containing pendant groups; and
(c) at least one linker between the polymeric backbone and each of the one or more pendant groups;
wherein the at least one linker comprises a hydrolyzable group.

Aspect 2. The charge-shifting polymer of aspect 1, wherein the hydrolyzable group comprises an amide, an ester, a thioester, or any combination thereof.

Aspect 3. The charge-shifting polymer of aspect 1 or 2, wherein the charge-shifting polymer has a number-average molecular weight (Mn) of from about 10,000 Da to about 24,000 Da.

Aspect 4. The charge-shifting polymer of aspect 3, wherein the charge-shifting polymer has an Mn of about 22,600 Da.

Aspect 5. The charge-shifting polymer of any one of aspects 1-4, wherein the charge-shifting polymer has a polydispersity of from about 1.00 to about 1.05.

Aspect 6. The charge-shifting polymer of aspect 5, wherein the charge-shifting polymer has a polydispersity of about 1.03.

Aspect 7. The charge-shifting polymer of any one of aspects 1-6, wherein the hydrolyzable group has a hydrolytic half-life of from about 15 hours to about 1800 hours at a temperature of from about 25° C. to about 50° C. and a pH of from about 5.5 to about 8.5.

Aspect 8. The charge-shifting polymer of any one of aspects 1-7, wherein the charge-shifting polymer is biocompatible.

Aspect 9. A composition comprising the charge-shifting polymer of any one of aspects 1-8 conjugated to at least one therapeutic molecule.

Aspect 10. The composition of aspect 9, wherein the at least one therapeutic molecule comprises a small molecule, a peptide, a protein, DNA, RNA, or any combination thereof.

Aspect 11. The composition of aspect 10, wherein the small molecule comprises coumarin, a coumarin derivative, camptothecin, doxorubicin, tetramethylrhodamine cadaverine, or any combination thereof.

Aspect 12. The composition of aspect 11, wherein the coumarin derivative comprises DMAE-modified 3-bromoacetyl coumarin (DBAC).

Aspect 13. The composition of aspect 12, wherein the at least one therapeutic molecule comprises two small molecules.

Aspect 14. The composition of aspect 13, wherein the two small molecules comprise DBAC and tetramethylrhodamine cadaverine.

Aspect 15. The composition of aspect 13 or 14, wherein the DBAC and tetramethylrhodamine cadaverine are present in a ratio of from about 99:1 to about 99.5:0.5.

Aspect 16. The composition of any one of aspects 9-15, wherein the hydrolyzable group comprises at least a first hydrolyzable group and a second hydrolyzable group, and wherein the first hydrolyzable group and the second hydrolyzable group have different hydrolytic half-lives under a single set of pH and temperature conditions.

Aspect 17. The composition of aspect 16, wherein the first hydrolyzable group has a hydrolytic half-life that is shorter than a half-life of the second hydrolyzable group.

Aspect 18. The composition of aspect 16 or 17, wherein the hydrolyzable group further comprises a third hydrolyzable group having a different hydrolytic half-life from both the first hydrolyzable group and the second hydrolyzable group under a single set of pH and temperature conditions.

Aspect 19. A method for controlled delivery of at least one therapeutic molecule to a subject, the method comprising administering the composition of any one of aspects 9-18 to the subject.

Aspect 20. The method of aspect 19, wherein hydrolysis of the charge-shifting polymer causes the at least one therapeutic molecule to disassociate from the charge-shifting polymer.

Aspect 21. The method of aspect 19 or 20, wherein hydrolysis of the charge-shifting polymer occurs intracellularly.

Aspect 22. The method of aspect 21, wherein hydrolysis of the charge-shifting polymer occurs in lysosomes.

Aspect 23. The method of aspect 20, wherein the hydrolyzable group comprises a thioester and the at least one therapeutic molecule dissociates from the charge-shifting polymer after uptake by a cell, wherein the cell comprises free glutathione.

Aspect 24. The method of aspect 23, wherein the free glutathione has a concentration in the cell of from about 1 to about 10 mM.

Aspect 25. The method of any one of aspects 19-24, wherein the composition is administered orally or intravenously.

Aspect 26. The method of aspect 25, wherein the composition is administered orally and wherein the charge-shifting polymer has a terminal half life of from about 5 to about 9 hours.

Aspect 27. The method of any one of aspects 19-26, wherein the subject is a mammal.

Aspect 28. The method of aspect 27, wherein the mammal is a human, mouse, rat, hamster, rabbit, guinea pig, cat, dog, sheep, goat, swine, cattle, or horse.

Aspect 29. A method for making a charge-shifting polymer, the method comprising contacting an azlactone polymer with an amine, wherein the amine comprises a structure of Formula I:

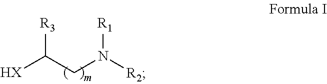

wherein m is from 0 to 10;
wherein X is selected from S, O, NH, or any combination thereof;
wherein R1 and R2 are independently selected from a linear or branched C1-C10 alkyl group or wherein R1, N, and R2 together form a 3- to 6-membered cycloalkyl or heterocycloalkyl group;
and wherein R3 is hydrogen or a linear or branched C1-C10 alkyl group.

Aspect 30. The method of aspect 29, wherein the compound of Formula I is:

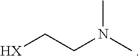

Aspect 31. The method of aspect 29 or 30, further comprising contacting the azlactone polymer and tertiary amine with a base.

Aspect 32. The method of aspect 31, wherein the base comprises 1,8-diazabicyclo(5.4.0)undec-7-ene (DBU).

Aspect 33. The method of any one of aspects 29-32, wherein the azlactone polymer comprises poly 2-vinyl-4,4-dimethylazlactone (PVDMA).

Aspect 34. The method of any one of aspects 29-33 wherein the method is conducted at about 45° C. for from about 24 to about 72 h.

Aspect 35. A charge-shifting polymer produced by the method of any one of aspects 29-34.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Temperature- and pH-Dependent Hydrolysis Rates of Tertiary Amine-Modified PVDMA with Amide, Ester, and Thioester Linkages

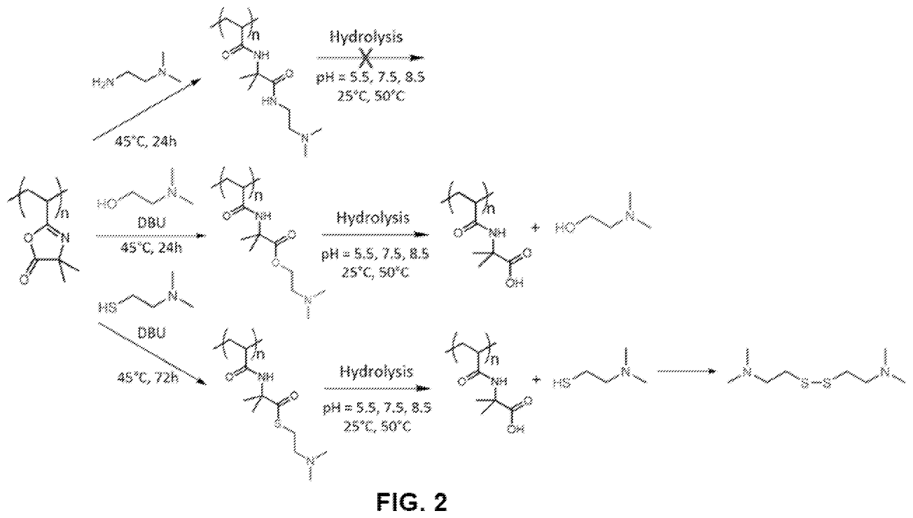

In order to investigate the charge-shifting capabilities of these materials, PVDMA was first modified with small-molecule nucleophiles that all contained a tertiary amine moiety along with unique nucleophilic reactive handle (either a primary amine, alcohol or a thiol moiety) that can then ring-open the azlactone ring. The resulting polymers were comprised of tertiary amine moieties connected to the polymer backbone via either an amide, ester or thioester linkage as depicted in FIG. 2.

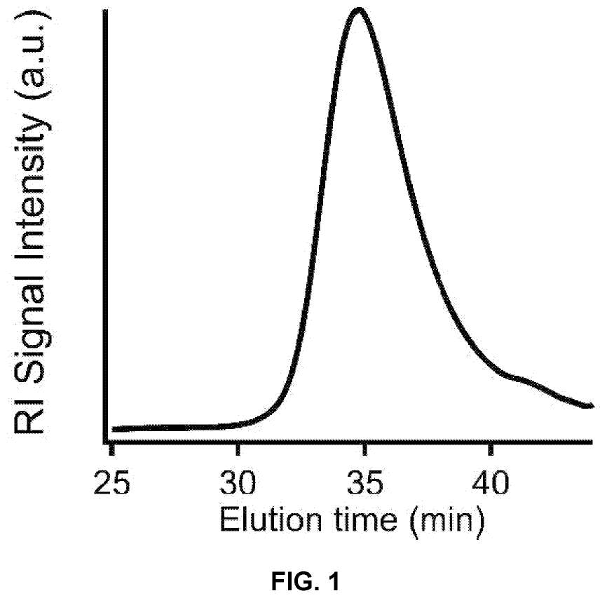

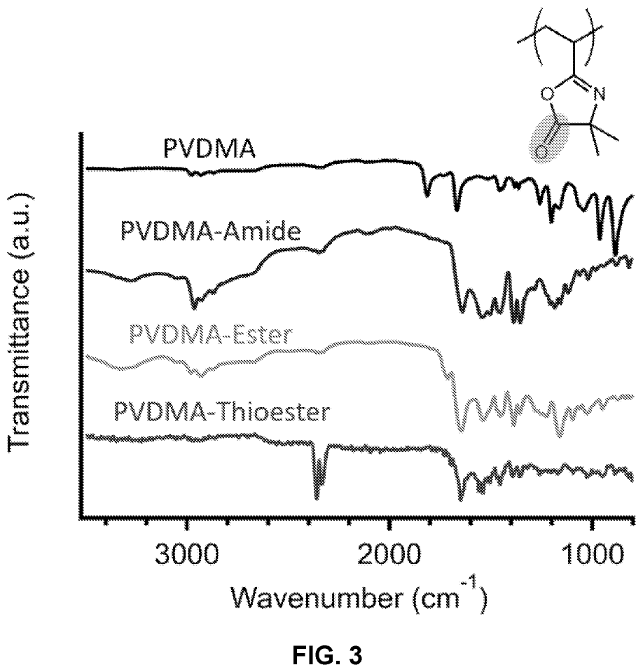

In order to study the hydrolytic degradation of PVDMA functionalized with side chains connected to the backbone via amide, ester, and thioester moieties, PVDMA was first prepared via RAFT polymerization using a similar procedure to previous literature reports. The number-average molecular weight (Mₙ) and dispersity (Ð) of the obtained PVDMA were analyzed via gel-permeation chromatography (GPC) and were found to be 22,600 g/mol and 1.03, respectively (FIG. 1). ¹H NMR spectroscopy was performed on this product, and the characteristic resonance peaks were analyzed. Since PVDMA can be utilized as a reactive platform, this product was functionalized with a small library of structurally-similar tertiary amine moieties bearing various nucleophilic reactive handles, including primary amine, hydroxyl, or thiol moieties (FIG. 2). The resulting modified polymers possessed side chains with tertiary amines connected to the backbone via amide bonds, ester bonds, and thioester bonds. The complete conversion of the azlactone was verified via Fourier transform infrared (FTIR) spectroscopy, and the spectra are shown in FIG. 3. As can be seen, the PVDMA precursor exhibited a strong absorbance band at 1810 cm⁻¹, which is characteristic of the azlactone carbonyl bond. Upon post-polymerization modification with each of the small molecule amines, this absorbance band disappeared, indicating complete conversion of the PVDMA scaffold into the functionalized product. ¹H NMR spectroscopy was used to identify the characteristic peaks associated with the modified PVDMA scaffolds.

In order to investigate the rate of hydrolysis of the PVDMA-derived charge-shifting polycations, hydrolysis experiments were conducted with each of the modified PVDMA species in pH 5.5, 7.5, and 8.5 buffered solutions at 25° C. and 50° C. The rates were monitored via ¹H NMR spectroscopy by observing the changes in the peaks associated with the side chains at various time intervals. If the functionalized polymer undergoes hydrolysis, the small-molecule tertiary amines located on the side chains are cleaved, resulting in carboxylic acids (FIG. 2) and, depending on the system's pH, could result in a full or partial shift of the net polymer charge from cationic to anionic.

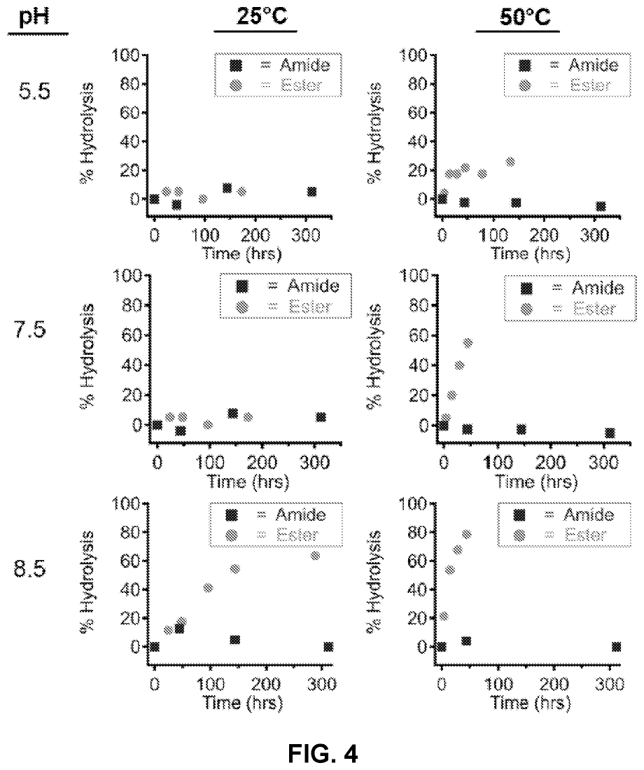

The kinetics of hydrolysis for the ester- and amide-modified PVDMA are shown in FIG. 4. At all pH values and temperatures, the hydrolysis rate of the PVDMA functionalized via an amide linkage was negligible over 300 hours. This is unsurprising as amide bonds are relatively stable and hydrolyze slowly compared to more labile bonds such as esters. The hydrolysis rates of the PVDMA functionalized via ester linkages exhibited a temperature and pH dependence, where the degradation rate increased with both increasing pH and temperature. This trend is in line with a previous report examining the hydrolysis rates of the structurally-similar polymer PDMAEA. The half-lives of ester-modified PVDMAs in various buffers are shown in Table 1. The pH 5.5 ester-modified sample at 25° C. did not exhibit an appreciable hydrolysis rate during the observation timeframe. However, the degradation half-life decreases to approximately 330 hours when the temperature is elevated to 50° C. The pH 7.5 samples showed degradation half-lives of approximately 1,800 hours and 40 hours at 25° C. and 50° C., respectively, and the pH 8.5 samples showed degradation half-lives of 225 hours and 30 hours at 25° C. and 50° C., respectively. These results demonstrate that the hydrolysis rates of ester-modified PVDMA can be tuned by varying both pH and temperature. The thioester-functionalized PVDMA exhibited pH- and temperature-dependent hydrolysis similar to the ester-functionalized PVDMA. Interestingly, the hydrolysis rate of thioester-functionalized PVDMA appears to be more rapid than ester-modified PVDMA.

TABLE 1
Temperature- and pH-dependent hydrolytic half-lives of tertiary
amine-functionalized PVDMA are connected via esters

| Temperature | Half-Life (hrs) | | |
|---|---|---|---|
| (° C.) | pH 5.5 | pH 7.5 | pH 8.5 |
| 25 | — | 1800 | 225 |
| 50 | 330 | 40 | 30 |

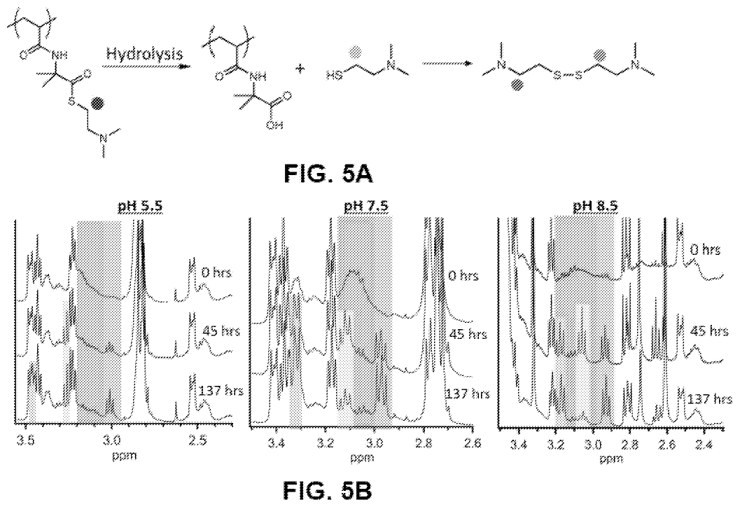

Unfortunately, the hydrolysis rate of thioester-functionalized PVDMA could not be accurately quantified via ¹H NMR spectroscopy owing to significant overlap between peaks associated with the functionalized polymer and small-molecule disulfide byproducts as shown in FIGS. 5A-5B for the 50° C. hydrolysis. However, thioester degradation can be monitored qualitatively over time by ¹H NMR spectra, as shown in FIG. 5B for the samples at 50° C. The resonance peak associated with the methylene protons adjacent to the thioester functionality on the polymer decreases. In contrast, the peak corresponding to the same hydrogen on the small molecule begins to appear in an overlapping region. By monitoring these peak changes, pH-dependent hydrolysis of the thioesters was again observed, where the thioester-functionalized PVDMA seems to be fully hydrolyzed by 15 hrs at 50° C. for both pH 7.5 and 8.5. In contrast, the hydrolysis rate of the thioester at pH 5.5 appears to be much slower, and complete degradation seems to take at least 137 hrs at 50° C. Interestingly, a side reaction occurs between the small-molecule thiol byproducts that becomes more prominent in pH 7.5 and 8.5, as evidenced by the disappearance of the resonance peaks associated with the small-molecule thiol with the simultaneous appearance of resonance peaks associated with the byproduct. These resonance peak shifts are consistent with the formation of disulfide bonds between the small-molecule thiols. To confirm the identity of this byproduct, ¹H NMR and ultraviolet-visible spectroscopy (UV-Vis) were performed on samples of the small-molecule thiol incubated in the various buffers at 50° C. and the structure was confirmed as a disulfide that forms between the small-molecule thiols as shown in FIG. 5A. The ¹H NMR spectra of the thioester-modified PVDMA samples incubated in pH 5.5, pH 7.5, and pH 8.5 buffer at 25° C. exhibit similar pH and temperature-dependent trends in hydrolysis rates. Although the hydrolysis rate for the thioester-modified PVDMA could not be quantified via ¹H NMR spectroscopy, a few interesting qualitative observations can be made. For example, the hydrolysis rate increases with increasing temperature and pH, the same trend as the ester-modified PVDMA.

Additionally, the thioester moiety appears to be almost fully hydrolyzed at pH 7.5 and 8.5 after 15 hrs at 50° C. These degradation rates appear similar to those shown by the ester-modified PVDMA, if not significantly faster. Since thioesters are considered more susceptible to nucleophilic attack than esters and thiolate anions are more stable than oxalate anions, it is hypothesized that the degradation of thioester-modified PVDMA will prove to be more rapid than esters which would extend the kinetic window for the charge transformation of PVDMA-based charge shifting polycations allowing for more rapid payload delivery in potential delivery applications. Furthermore, the degradation of PVDMA modified with thioester linkages may proceed even more rapidly in a cellular environment that is high in concentrations of thiols like glutathione, which has been demonstrated to cleave thioester linkages more rapidly than hydrolysis alone. This could be intriguing for applications requiring rapid payload delivery whereby amine-modified PVDMA with thioester linkages could deliver negatively-charged biomolecules to cellular environments in minutes to hours.

In conclusion, the pH- and temperature-dependent hydrolysis rates of tertiary amine-modified PVDMA with amide, ester, and thioester linkages were investigated. While prior studies mainly focused on controlling the hydrolysis rate of PVDMA-derivatives by changing the nature of the linker moieties from either ester or amide, this chemistry was extended to include the thioester linker. Additionally, a systematic study of the influence of pH and temperature on the hydrolytic degradation of these various linker moieties was performed. While the amide linkages showed no detectable hydrolysis over 300 hours for any of the pH and temperatures investigated, the ester and thioester linkages exhibited increasing hydrolysis rates with both increasing temperature and pH. The ester-modified PVDMA exhibited hydrolytic half-lives that varied from over 1,800 hrs at pH 7.5 and 25° C. to 30 hrs at pH 8.5 and 50° C., suggesting that the degradation (and hence the rate of charge shifting) of these materials can be tuned via temperature and pH. The thioester-modified PVDMA hydrolysis rates also increased with both temperature and pH and appeared to be faster than esters, although further quantification is needed to confirm this observation. These results can be utilized for the future design of PVDMA-based charge-shifting polymers by offering temperature- and pH-dependent hydrolysis rates and introducing a novel thioester-based charge-shifting polycation that appears to degrade more rapidly than its ester counterpart. These tunable hydrolysis rates could enable applications such as the delivery of bioactive agents that require precise control of the hydrolysis in timeframes varying from minutes to hours.

### Example 2: The Modification of PVDMA with Tertiary Amine Moieties Via Thioester Linkages and Glutathione-Triggered Release of DNA

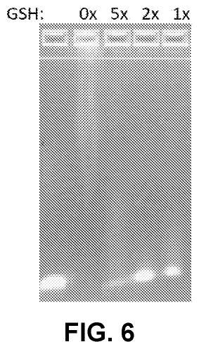

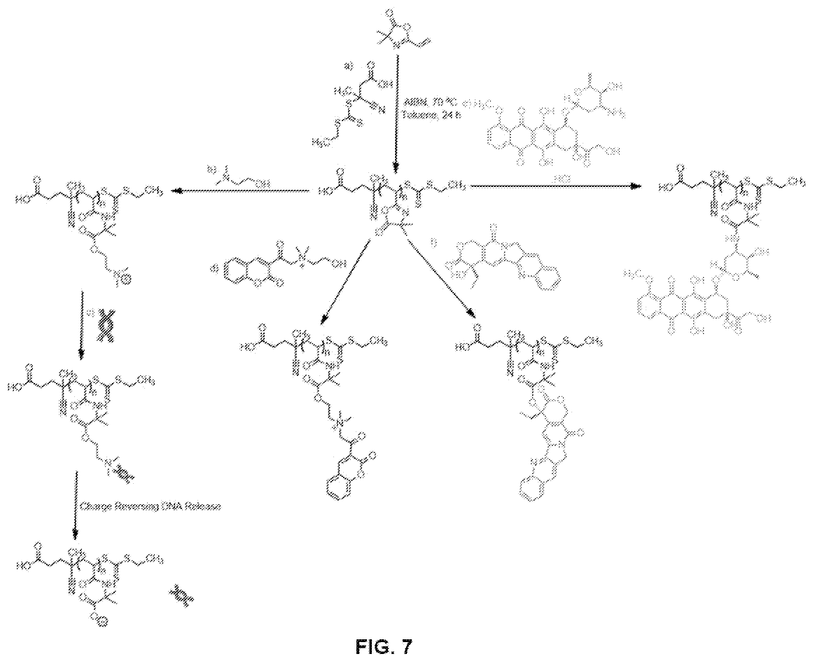

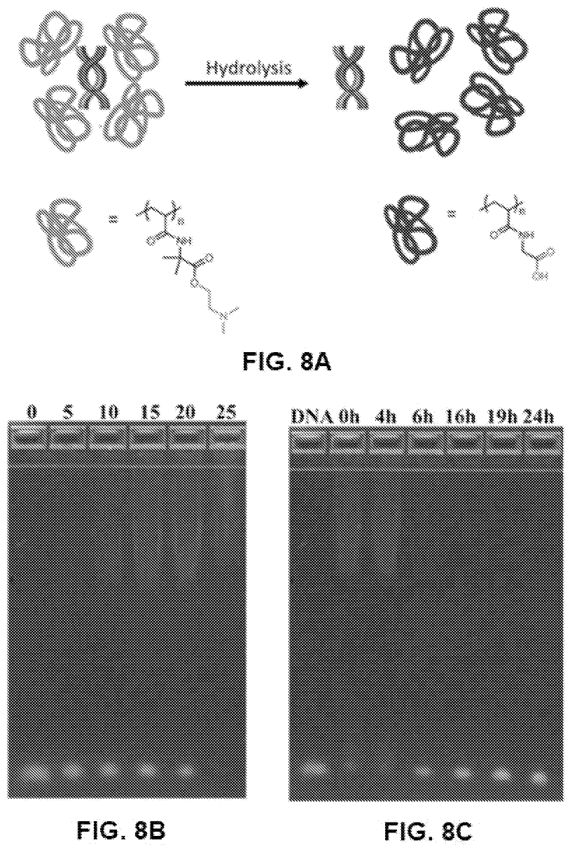

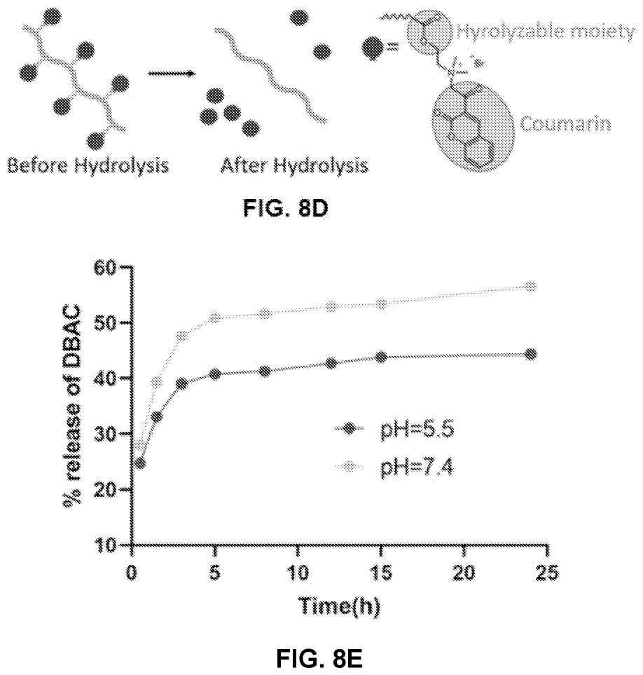

In addition to PVDMA modified with ester moieties which show hydrolysis-induced release of small molecule drugs and nucleic acids, tertiary-amine counterparts were prepared that were connected to the polymer backbone via thioester linkages. Since thioesters can undergo rapid thiol-thioester exchange reactions, it was hypothesized that this would cause the release of the nucleic acid cargo once the polyplexes were internalized into cells since the intracellular concentration of glutathione (which possesses a free thiol moiety) is relatively high (1-10 mM). The agarose gel electrophoresis results are shown in FIG. 6. The left-most cell is “free DNA” formulated without any polymer. The rest of the cells are polyplexes formulated at N/P ratios of approx. 20/1 with varying ratios of glutathione (GSH) introduced into the formulations. As shown in FIGS. 8A-8D, as GSH was introduced into the polyplex formulations, it triggered the release of DNA from the polyplexes. These results are indicative that the tertiary amine-functionalized PVDMA with thioester linkages could exhibit an intriguing “triggered release” of the nucleic acids once internalized into cells.

### Example 3: Modification of PVDMA with Other Small Molecules to Prepare Stimuli-Responsive Polymer-Drug Conjugates

After investigating the relative rates of hydrolysis of the cationic PVDMA-derivates, small-molecule drugs were conjugated to PVDMA via ester linkages as shown in FIG. 7. The drugs investigated were coumarin, doxorubicin, and camptothecin. Additionally, tertiary-amine modified PVDMA was prepared to investigate its ability to complex and release nucleic acids upon the hydrolysis of the ester linkages. As shown in FIG. 8B, the tertiary amine-modified PVDMA can complex DNA at N/P values of 25. FIG. 8C shows the hydrolysis-induced release of DNA from the polyplexes formulated at N/P ratios of 25 and incubated at pH 7.4. The results indicate that DNA is released from the polyplexes over the course of approx. 6 h. The release of the small-molecule coumarin model drug at pH 5.5 and 7.4 are shown in FIG. 8E and demonstrate an initial burst-release followed by a steady release over several hours. These results demonstrate that PVDMA can be utilized to prepare materials that can deliver drugs and nucleic acids in a time-released manner.

The delivery of coumarins from the coumarin-modified PVDMA polymer (FIG. 8D) in cells and living animals was further investigated to confirm the utility of the disclosed compositions and methods. Importantly, this is the first instance of a PVDMA-based polymer-drug conjugate, the first in-depth investigation of intracellular trafficking of PVDMA-based polymer-drug conjugates, and the first time using a PVDMA-based delivery approach in vivo.

### Materials

All materials were used as received without further purification unless noted otherwise. 2-Vinyl-4dimethyl azlactoneone (VDMA) purchased from (Polysciences, USA). RAFT agent 4-Cyano-4-(((ethylthio)carbonothioyl)thio)pentanoic acid (ECT) and initiator 2,2′-Azobis(2-methylpropionitrile) (AIBN), 3-bromoacetyl coumarin (BAC), 2-Dimethylaminoethanol (DMAE), 1,8-diazabicyclo[5.4.0]-undec-7-ene (DBU), 4-Aminophenyl disulfide (APD), L-Glutathione reduced (GSH), Triethylamine (TEA), Potassium phosphate monobasic, Potassium phosphate dibasic, Trizma Base, Trizma HCl all of the chemicals were purchased from (Sigma-Aldrich, USA). Tetramethylrhodamine Cadaverine (TMR) and Phosphate-Buffered Saline (pH 7.4), E-Gel EX Agarose Gels (2%) were purchased from (Thermofisher Scientific, USA). Dialysis bag MW cut off 3.5 KDa was purchased from (Fisher Scientific, USA). Doxorubicin Hydrochloride was purchased from TCI chemical, USA. Camptothecine drug procured from selleckchem.com, USA. Chloroform-d (CDCl3), Methanol-d4, deuterium oxide (D2O), N, N-Dimethylsulfoxide-d6 all the NMR solvents, were purchased from (Sigma-Aldrich, USA). All the solvents toluene, acetone, diethyl ether, N, N-dimethylformamide (DMF), dimethyl sulfoxide (DMSO), methanol, acetonitrile, were purchased from (Fisher chemicals, USA). Scrambled DNA was purchased from IDT.

### Characterization

Nuclear magnetic resonance (NMR) spectroscopy. ¹H NMR of all the synthesized (co)polymers were recorded at 25° C. on a Bruker 400 MHz spectrometer using CDCl₃, D₂O, MeOD, or DMSO-de as the solvent.

Attenuated total reflection infrared (ATR-IR) spectroscopy. IR Spectra of the (co)polymers were recorded on a Perkin Elmer Spectrum 100 in the attenuated total reflectance (ATR) mode with a diamond crystal, using a spectral range of 4000-500 cm⁻¹.

Gel permeation chromatography (GPC). Chromatography was performed on an Agilent 1260 Infinity II LC System with degasser, pump, autosampler, and UV detectors. GPC analyses were done using two serial Tosoh Biosciences TSKGel Alpha columns (25000 and 3000) (Tokyo, Japan) with dimethylformamide modified with 0.01 LiBr as the mobile phase at a flow rate of 0.5 mL/min. Wyatt miniDAWN TREOS light scattering detector and Opti-TrEX refractive index detector (Wyatt Technology Corp., Santa Barbara, CA, USA) were used to obtain absolute molecular weights.

Microplate reader. Fluorescence and luminescence measurements were gathered using a BioTek Synergy H1 microplate reader.

UV-Vis spectroscopy. Absorbance of DMAE-conjugated BACs (DBACs) was measured using a Thermo Scientific Evolution 201 UV-Visible Spectrophotometer.

Confocal microscope. Super-resolution fluorescent cellular imaging was performed on a Leica Stellaris stimulated emission depletion (STED) confocal microscope.

UPLC-MS/MS. Chromatography was performed on an Acquity™ UPLC system (Waters Corp, Milford, MA) with an autosampler temperature at 10° C. Waters Acquity UPLC® BEH Phenyl column (3.0×50 mm, 1.7 μm particle size) was used for chromatographic separation with linear gradient elution consisting of (A) 90% acetonitrile and (B) 10% of 0.2% formic acid in Milli-Q water as mobile phases. The flow rate was set at 0.30 mL/min, and the injection volume was 2 μL.

An Acquity Tandem Quadrupole Mass Detector (Xevo TQ-S; Waters Corp, Milford, MA) in positive electrospray ionization mode was used for mass spectrometric detection. For collision-induced dissociation, argon was used as collision gas. The cone voltage and collision energy were set at 40 V and 24 V for DBAC and 40 V and 26 V for the tolbutamide (IS), respectively. Quantification was performed using the monitoring of multiple reaction monitoring (MRM) of following transitions: m/z 276/186 for DBAC and m/z 271/90.9 for IS. Retention times of DBAC and IS were 1.22 and 1.46 minutes, respectively.

### Methods

Homopolymerization of (2-vinyl-4,4-dimethyl azlactone) via reversible addition-fragmentation chain transfer (RAFT) polymerization. The reaction was performed in a single-neck pear-shaped 50 mL round-bottom reaction flask equipped with a Teflon-coated magnetic stir bar. VDMA monomer (8.0 g, 57.5 mmol), ECT (84.3 mg, 0.32 mmol), AIBN (5.25 mg, 0.032 mmol) and toluene (24 mL) were added into the round-bottom flask. The round-bottom flask was sealed with a rubber septum and purged with nitrogen for 30 minutes. The reaction mixture was placed in a preheated oil bath at 70° C. for 24 h. Finally, the reaction was quenched by placing a round-bottom flask into an ice bath. The product was isolated by two precipitations in chilled cold diethyl ether and dried in vacuo. ¹H NMR (400 MHz, CDCl3, δ in ppm): 1.55-1.30 (O═C—C—(CH₃)₂ and —CH₂—CH₃ signal a), 2.18-1.82 (—CH₂CH, signal d), 2.84-2.60 (—CH₂CH, signal C). FT-IR (ATR, cm-1): 2975 (4,4-dimethyl groups on the azlactone ring), 2933 and 2867 (C—H str. of methylene and methine backbone signals), 1810 (lactone C═O) and 1668 (C═N).

Synthesis of N-(2-hydroxyethyl)-N,N-dimethyl-2-oxo-2-(2-oxo-2H-chromen-3-yl)ethanaminium (DBAC). The DBAC was synthesized by following a previously reported procedure. 3-bromoacetyl coumarin (0.5 g, 1.87 mmol) and 2-dimethylaminoethanol (2.0 mL, 19.87 mmol) were taken in a 15-mL pear-shaped flask and vortexed vigorously. After that, the reaction vessel was placed in a preheated oil bath at 40° C. and stirred for 2 hours. The reaction mixture was allowed to cool to room temperature, then added dropwise into acetone (this process was done three times), and a brown precipitate was obtained. The precipitate was collected by filtration and dried under vacuum. ¹H NMR (400 MHz, D₂O, δ in ppm): 2.83-2.73 (—CH₂—OH, signal a), 3.51-3.42 and 3.22-3.13 (—CH₂—N(CH₃)₂, signal d and e), 3.62-3.53 (—N—CH₂—CH₂, signal c), 4.19-3.73 (—CH₂—CH₂—OH, signal b), 4.64-4.46 (O═C—CH₂. signal f), 7.42-7.28 (—CH of benzene, signal i), 7.74-7.54 (—CH of benzene, signal h), 8.38-8.25 (O═C—C═CH, signal g). FT-IR (ATR, cm-1): 3125, 3031 and 3014 (C—H vibrations of 3-bromoacetyl coumarin), 1716 (C═O stretching frequency of 3-bromoacetyl coumarin), 1606 and 1577 (ring vibration which is localized on the benzene part of the molecule), 3395 (OH stretching of DMAE).

Post polymerization modification of PVDMA by DBAC. The five-member ring of PVDMA was opened by the following procedure; in a 15-mL pear-shaped flask, PVDMA (150 mg, 1.07 mmol with respect to monomer repeat unit) was taken and dissolved in 2 mL of DMF. DBAC (595 mg, 2.154 mmol) was separately dissolved in 5 mL of DMF in another vial. After the solutions were mixed, DBU (2.1 μL, 0.014 mmol) was added. The reaction was allowed to run for 24 hours at 50° C. The product was subsequently precipitated in chilled diethyl ether (the precipitation process was done three times). Precipitate was collected by filtration and dried under vacuum. ¹H NMR (400 MHz, MeOD, δ in ppm): 1.60-1.50 (—NH—C(CH₃)₂, CN—C—CH₃ and —CH₂—CH₃, signal a, b and c), 2.46-2.12 (—CH₂CH and COOH—CH₂, signal d and e), 3.59 and 3.27 (—CH₂—N(CH₃)₂, signal f and g), 3.37 (—CH₂—CH, signal h), 3.64-3.63 (—O—CH₂—CH₂, signal i), 4.77-4.57 (—N—CH₂—C═O, signal j), 4.26-4.12 (—O—CH₂—CH₂, signal k), 7.47-7.34 (—CH of benzene, signal n), 7.77-7.66 (—CH of benzene, signal m), 8.04-7.89 (—C═O—NH, signal o), 8.37-8.31 (—CH—C—C═O, signal l). FT-IR (ATR, cm 1): 1714 (C═O stretching frequency of ester), 1646 (C═O stretching of amide), 1535 (N—H bending of amide), 2975 (4,4-dimethyl groups on the azlactone ring), 2933 and 2867 (C—H str. of methylene and methine backbone signals), 3300 (N—H stretching of amide).

Functionalization of PVDMA with DMAE. PVDMA (100 mg, 0.72 mmol with respect to the molecular weight of the polymer repeat unit) was dissolved in 3 mL anhydrous DMF and 1.5 equivalents of DMAE (dimethylaminoethanol) and 2 equivalents of DBU were added to the reaction flask. The resulting solution was stirred at 50° C. for 48 hours. The polymer was then precipitated into a hexane/acetone solution (30 mL, 4:1 v/v) and the polymer was collected by decanting the supernatant. The polymer was redissolved into THF and precipitated two more times. The produce was dried for 24 hours under a vacuum.

Post-polymerization modification of PVDMA by anti-cancer drugs. PVDMA polymer (50 mg, 0.36 mmol) and doxorubicin-HCl (53 mg, 0.09 mmol) were taken in a 10 mL pear-shaped flask and dissolved in 1.5 mL DMF solvent. Further, triethylamine (TEA) (13 μL, 0.09 mmol) was added to the mixture and then continued stirring at room temperature for 24 h under dark conditions. After that, Doxorubicin-modified PVDMA polymer was purified by dialysis against DMF and then water with 3.5 KDa cut off dialysis bag. The recovered sample was then frozen and lyophilized. ¹H NMR (400 MHz, DMSO-d₆, δ in ppm): 1.48-1.11 (—NH—C(CH₃)₂, —CH₂—CH₃, and —O—CH₂—CH₃, signal a, b and c), 1.92-1.77 (—CH—CH₂, signal d), 2.34-2.15 —CH—CH₂, signal e), 3.13-2.99 (—C—CH₂—C, signal f), 4.05-3.88 (—O—CH₃, signal i), 4.28-4.10 (—O—CH—CH₃, signal j), 4.71-4.48 (—C═O—CH₂—OH, signal k), 5.07-4.76 (—CH₂—CH—O—, signal g), 5.54-5.13 (—O—CH—CH₂, signal h), 6.41-6.01 (—OH of doxorubicin), 7.82-7.33 (benzene ring of doxorubicin), 8.23-7.87 (—NH of amide and doxorubicin), 14.18-13.84 and 13.41-13.07 (—Ar—OH of doxorubicin). FT-IR (ATR, cm⁻¹): 1720 (C═O stretching frequency of doxorubicin), 1652 (C═O stretching of amide), 1533 (N—H bending of amide), 1411 (doxorubicin str.), 1284 (doxorubicin str.), 1016 and 985 (doxorubicin str.), 792 and 763 (doxorubicin str.).

PVDMA polymer (50 mg, 0.36 mmol) and camptothecin (125.5 mg, 0.36 mmol) were taken in a 10 mL pear-shaped flask and dispersed in 5 mL DMF solvent. Further, DBU (54 μL, 0.36 mmol) was added to the mixture and purged with nitrogen for 30 minutes. Next, the reaction mixture was placed into a preheated 40° C. oil bath with stirring for 24 h. After that, camptothecin-modified PVDMA polymer was purified by dialysis against DMF and then water with 3.5 KDa cut off dialysis bag. The recovered sample was then frozen and lyophilized. ¹H NMR (400 MHz, DMSO-d₆, δ in ppm): 0.94-0.83 (—C—CH₂—CH₃, signal a), 1.74-1.12 (—NH—C(CH₃)₂, —CH₂—CH₃, and —CH₂—C—CH₃, signal b, c and d), 2.38-1.97 (—C—CH₂—CH₃, signal e), 2.77-2.62 (—CH₂—CH, signal f), 3.29-3.21 (—CH₂—CH₃, signal g), 5.35-5.16 (—C═O—N—CH₂, signal i), 5.51-5.37 (—C═CH, signal j), 6.67-6.45 (camptothecine), 7.39-7.34 (—C═CH, signal h), 8.24-7.63 and 8.77-8.55 (benzene ring of camptothecine). FT-IR (ATR, cm⁻¹): 1735 (C═O stretching frequency of ester), 1579 and 1155 (camptothecine str.).

In vitro drug release studies. DBAC release studies were carried out in different pH (5.5 and 7.4) 0.3 M buffer solutions. DBAC-modified PVDMA polymer (5 mg) was dissolved in 1 mL buffer solution and poured into 3.5 kDa MW cut off dialysis bag. At 37° C., dialysis was performed against a 100 mL volume buffer solution. Over time, supernatants were gathered from the buffer solution and measured via UV-VIS spectrophotometry. Absorbance was measured at 316 nm for wide range of concentrations of DBAC, including 0.005, 0.010, 0.015, 0.020, 0.025, 0.030 and 0.035 mg/mL. Absorbance readings at 316 nm were used to generate a linear standard curve of absorbance vs. concentration.

Polyplex formation and gel electrophoresis with PVDMA-based polyplexes. DNA/polymer complexes were formed by adding 20 μL of a scrambled DNA solution (0.0028 mM in DI Water) to a solution of polymer (20 μL dissolved in 20 mM acetate buffer, pH=5.0) to yield the desired polymer/DNA N/P ratio. The pH of the solution was adjusted to 7.4 by adding 27.24 μL of Trizma buffer (20 mM, pH=8.5) and the polyplex solutions were incubated at 37° C. for various time points. Prior to analysis, the polyplex solutions were dilute with 67.24 μL of DI water. The polyplexes were analyzed on a 2% E-gel EX agarose gel by loading 20 μL of each polyplex solution per well.

Cell culture. Cancer cells (HeLa) and human embryonic kidney cells (HEK-293) (ATCC, USA) were cultured in EMEM (Corning, USA) and DMEM (Gibco) media, respectively, each containing 10% FBS (Gibco) and 1% Anti-Anti (100×) reagent (Gibco). All cell lines were maintained at 37° C. and 5% CO₂.

Cell viability. HeLa and HEK-293 cells in culture media were seeded in three replicates in a 96-well plate at a density of 3,000 cells/well. The cells were allowed to adhere and proliferate for 24 hr before replacing the media with new media containing varying concentrations of polymer solution (0-1000 μg/mL). The cells were incubated with the new media for 24 hr, after which the CellTiter Glo™ (Promega, Cat. No. G9242) assay was performed, and the luminescence of the cells was measured on a microplate reader (BioTek Synergy H1).

Synthesis of both DBAC and tetramethyl rhodamine (TMR) cadaverine-modified PVDMA polymer. The synthesis of fluorescently-labeled derivatives of PVDMA was performed by slight modification of a previous report. PVDMA (150 mg, 1.078 mmol w.r.t monomer repeat unit), DBAC (296 mg, 1.07 mmol), and DBU (4 μL, mmol) were taken into a 10 mL round-bottomed flask and dissolved in 4 mL of DMSO. In a separate vial TMR (2.77 mg, 0.005 mmol) was dissolved in 1 mL DMSO, and transferred into the mixture in the round-bottom flask. The flask was capped with a septum and stirred in the dark at 50° C. for 24 h. The solution was precipitated dropwise into 20 mL of cold diethyl ether, filtered, and dried under vacuum. ¹H NMR (400 MHz, DMSO-d₆, δ in ppm): 1.55-1.18 (—NH—C(CH₃)₂, signal a), 2.78-2.73 (—CH₂CH, signal b), 3.49 and 3.21 (—C—N(CH₃)₂, signal c and d), 3.44 (TMR), 3.57 (—O—CH₂—CH₂, signal e), 4.64-4.31 (—O—CH₂—CH₂, signal f), 4.19-4.06 (—N—CH₂—C═O, signal g), 7.58-7.17 (—CH of benzene and TMR, signal h), 7.75-7.59 (—CH of benzene, signal i), 7.97-7.86 (TMR), 8.33-8.25 (—C═O—C═CH, signal j). FT-IR (ATR, cm⁻¹): 1714 (C═O stretching frequency of ester), 1646 (C═O stretching of amide), 1535 (N—H bending of amide), 2975 (4,4-dimethyl groups on the azlactone ring), 2933 and 2867 (C—H str. of methylene and methine backbone signals), 3300 (N—H stretching of amide), 701 (benzene derivative).

Measurement of fluorescently-labeled PVDMA at various concentrations. For making a standard curve of TMR-modified polymer fluorescence, the fluorescence intensity of DBAC-TMR-PVDMA was measured using a microplate reader (BioTeK synergy H1) at an excitation wavelength of 544 nm and emission wavelengths of 575 nm. Measurement of TMR and DBAC modified PVDMA polymer was started in various concentrations ranging from 3.7 mg/ml to 0.0003 mg/mL by dissolving in pH 7.4 PBS buffer.

Intracellular trafficking of DBAC-TMR-PVDMA imaged via confocal microscopy. HEK-293 cells were utilized for intracellular imaging. The cells were grown under standard conditions (37° C., 5% CO₂, DMEM media with 10% FBS) in glass bottom culture vessels. 10 μg mL⁻¹ of DBAC-TMR-PVDMA polymer was added to the cells and incubated for 10 minutes or ON, after which fluorescence associated with dyes was imaged. In addition, time lapse imaging of dye distribution in cells was taken for 3 hours after adding 100 μg mL⁻¹ of the dye to cells in glass bottom culture vessels.

Subcellular colocalization imaging of DBAC-TMR-PVDMA. HEK-293 cells were grown under standard conditions (37° C., 5% CO₂, DMEM media with 10% FBS) in glass bottom culture vessels. Imaging of DBAC-TMR-PVDMA distribution in cells was done after a 2 h incubation with 50 μg mL⁻¹ of DBAC-TMR-PVDMA polymer in cell culture media. Cells were co-stained with LysoTracker Green DND-26 (Invitrogen) 10 mins before imaging, and washed with 1 mL PBS. Fluorescence and colocalization of dyes was visualized and measured with a Leica Stellaris STED confocal microscope.

Pharmacokinetics of DBAC-TMR-PVDMA. All procedures were performed in compliance with the University of Mississippi Animal Care and Use Committee and following the National Institutes of Health (NIH) guidelines. Male and female CD-1 mice (50:50 male:female ratio) were quarantined in the animal house of University of Mississippi for a period of 7 days with a 12 h dark/light cycle and during this period they had free access to standard pellet feed and water. Either DBAC or DBAC-TMR-PVDMA were administered at 10 mg/kg through oral gavage or retro-orbital IV injection at a volume of 0.1 mL. The vehicle for DBAC was 10% absolute alcohol, 10% cremophor and 80% Milli-Q water, whereas the DBAC-TMR-PVDMA polymer was administered in 100% PBS. At different times post-injection (0.5, 1, 3, 6, 8 and 15 h for oral and 0.12, 0.25, 0.5, 1, 3, and 8 h for IV) mice were induced with 5% isoflurane and maintained with 2% isoflurane (Covetrus, #029405). Blood was collected by cardiac puncture and stored in K2 EDTA tubes (SAI Infusion Technologies, #MVCB-E-300). Mice were then perfused through heart with PBS (Life Technologies, #10-010-072). Plasma was harvested by centrifuging the blood using Eppendorf 5430R Centrifuge (Germany) at 5,000 rpm for 5 min and stored frozen at −80±10° C. until analysis. Following the collection of GI tract, heart, lung, spleen, liver or kidney tissues in a separate 15 mL round-bottom screw-capped vial, phosphate buffered saline (5 volumes of each tissues) was added and homogenated with a homogenizer (Polytron®) and stored at −80±10° C. until analysis.

Sample preparation for DBAC detection. A simple protein precipitation method was followed for extraction of DBAC from mice plasma. To an aliquot of 50 μL of plasma or tissue (GI tract, heart, lung, spleen, liver or kidney) samples, tolbutamide (internal standard, IS) solution (5 μL of 20 ng/ml) was added and mixed for 15 s on a cyclomixer (Thermo Scientific, IN, USA). After precipitation with 500 μL of acetonitrile, the mixture was vortexed for 2 min, followed by centrifugation for 10 min at 14,000 rpm on an accuSpin Micro 17R (Fisher Scientific, USA) at 5° C. An aliquot of ˜150 μL of clear supernatant was transferred into vials and 2 μL was injected into the UPLC-MS/MS system for analysis. The primary stock solutions of DBAC and tolbutamide (IS) were prepared in methanol at 1.0 mg/mL concentration. Working solutions of calibration standards and quality control (QC) samples were prepared by dilution with methanol and stored at −20° C. A working stock of the IS solution (20 ng/ml) was prepared in methanol and stored at −20° C.

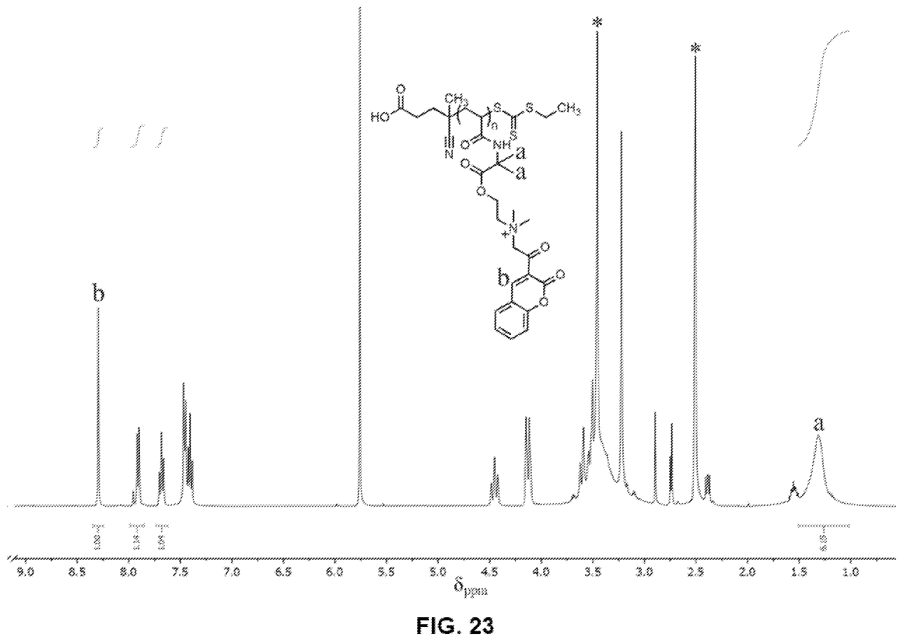

Sample preparation for fluorescent detection of DBAC-TMR-PVDMA in plasma and other tissues. 0.1 mL aliquots of all tissues were taken (GI tract, Liver, Heart, Kidney, Spleen and Lungs) except Plasma (20 μL added in 40 μL pH 7.4 PBS buffer) for measuring the fluorescence intensity of DBAC-TMR-PVDMA polymer. A microplate reader (BioTeK synergy H1) was used to measure the fluorescence intensity at an excitation wavelength of 544 nm and emission wavelengths of 575 nm. The concentration of fluorescently-labeled polymer in plasma and other tissues was calculated based on a standard curve in FIG. 23.

### Synthesis of PVDMA Homopolymer and DMAE-Modified 3-Bromoacetyl Coumarin (DBAC)

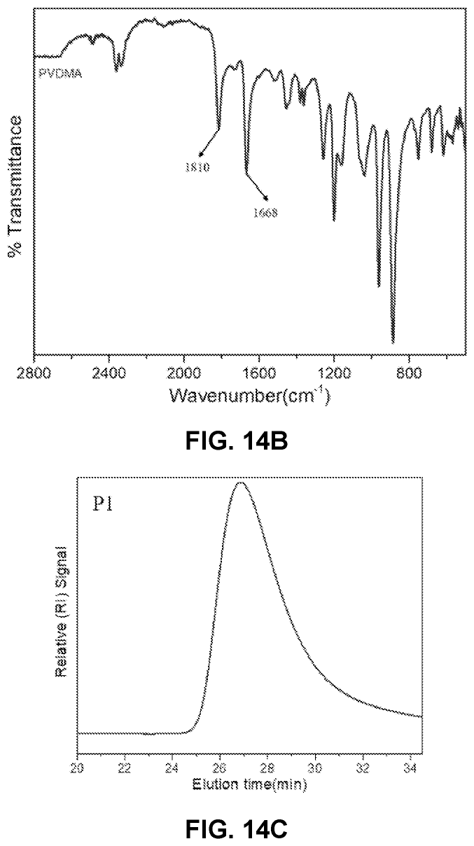

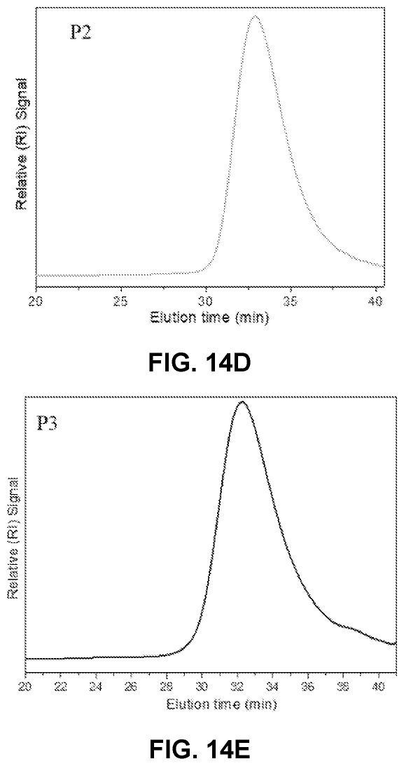

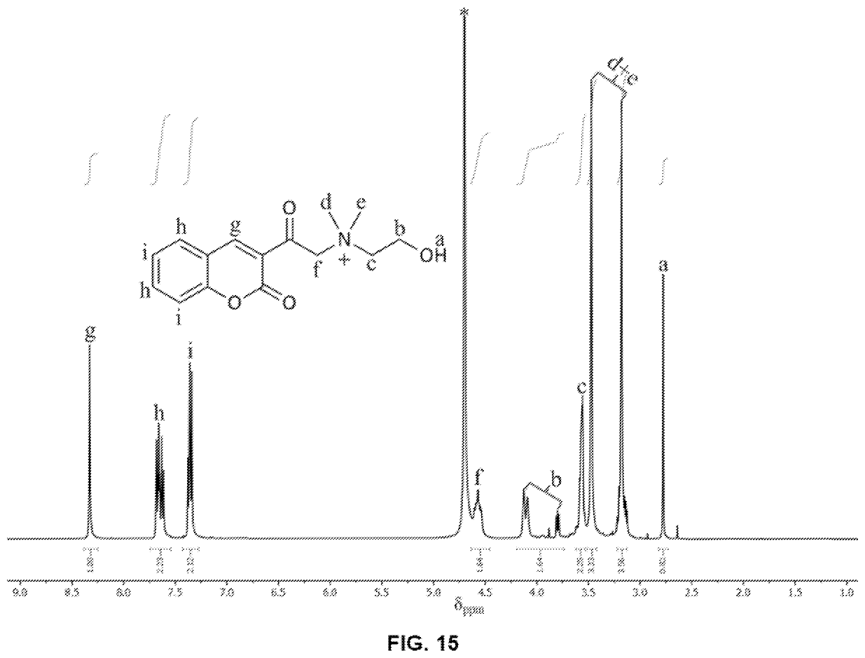

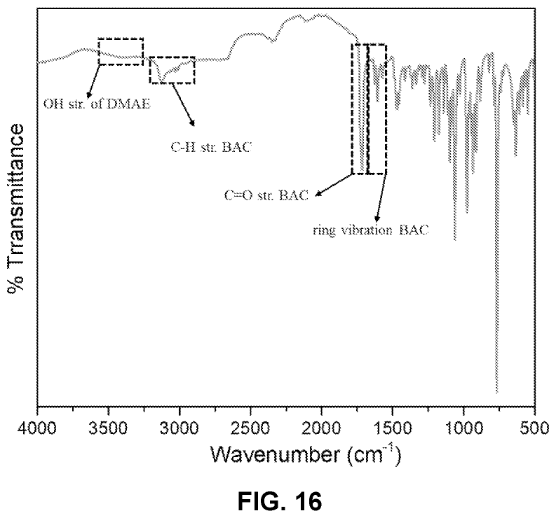

RAFT polymerization was used to synthesize well-defined and narrowly dispersed PVDMA homopolymers of three molecular weights (Scheme 1a and FIGS. 14A-14E). The structural characterization of PVDMA homopolymers was confirmed by ¹H-NMR (FIG. 14A) and FTIR spectroscopy (FIG. 14B). In ¹H NMR spectra, the presence of azlactone ring-associated methyl groups was identified at the peak at 1.55-1.30 ppm, while the peaks at 2.18 and 2.84 correspond to the polymer backbone. Moreover, FTIR spectroscopy revealed C═O stretching at 1810 cm⁻¹ and C═N stretching at 1668 cm⁻¹ of the azlactone ring. Gel permeation chromatography (GPC) results verified that PVDMA could be synthesized with varying molecular weights (P1=10,440 Da, P2=15,460 Da, and P3=23,180 Da) and consistently produce polymers of low polydispersity (P1=1.03, P2=1.01, and P3=1.03) (FIGS. 14C-14E, Table 2). Next, DBAC was produced by a tertiary amine alkylation reaction between BAC and DMAE (Scheme 2), followed by confirmation by ¹H-NMR (FIG. 15) and FTIR spectroscopy (FIG. 16).

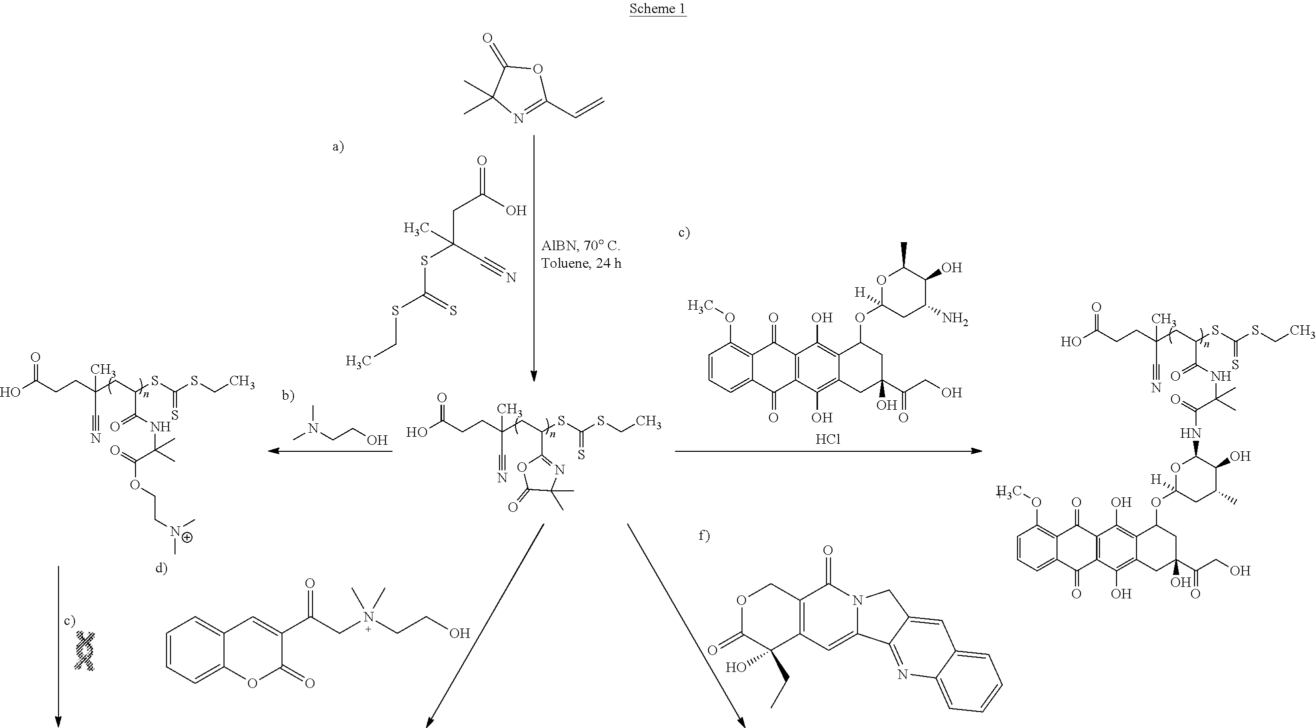

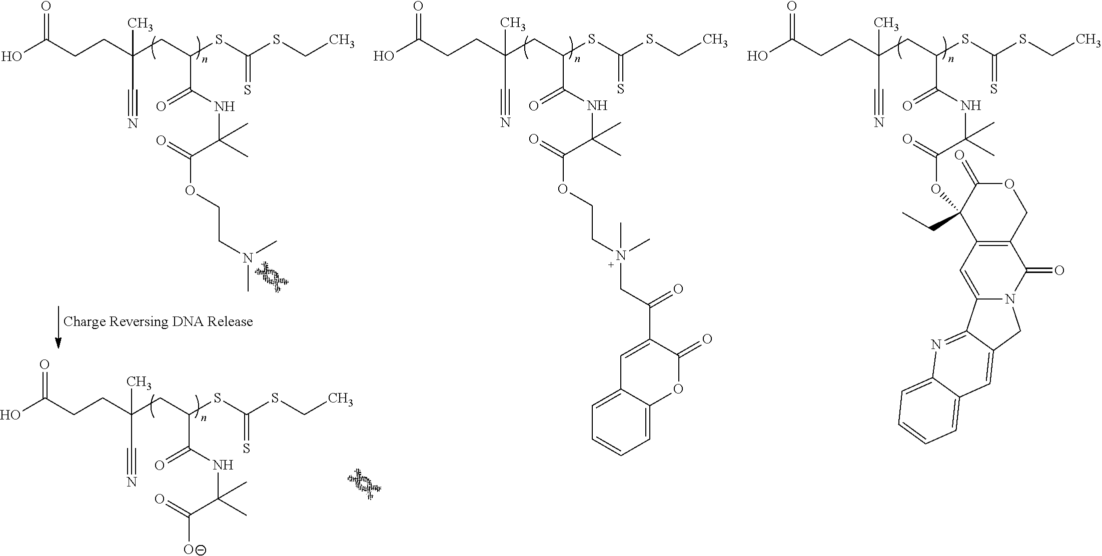

Scheme 1: Synthetic pathways for a) the PVDMA polymer scaffold, b) Post-polymerization modification of PVDMA with DMAE to form ester-PVDMA, c) Polyplex formation with ester-PVDMA binding a nucleic acid, d) Post-polymerization modification of PVDMA scaffold by DBAC, e) Post-polymerization modification of PVDMA by doxorubicin, and f) Post-polymerization modification of PVDMA by camptothecin.

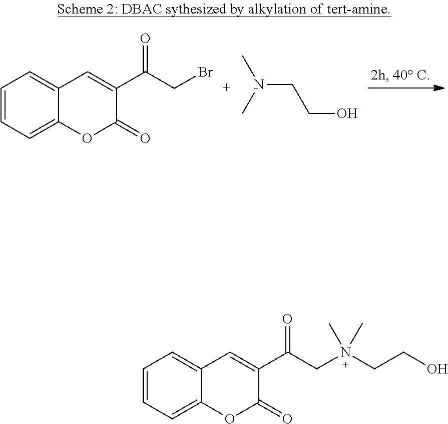

TABLE 2
Gel Permeation Chromatography Results
for Different PVDMA Polymers

| PVDMA | Molecular | Polydispersity |
|---|---|---|
| Polymer | Weight (g/mol) | Index (PDI) |
| P1 | 10,440 | 1.027 |
| P2 | 15,460 | 1.0112 |
| P3 | 23,180 | 1.026 |

Successful Functionalization of PVDMA Polymer with Drug Molecules

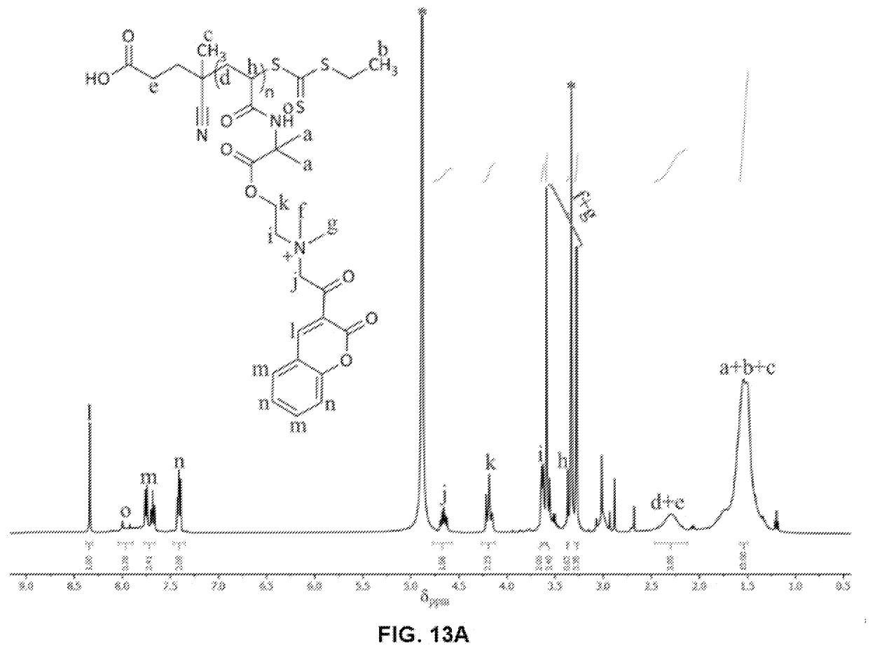

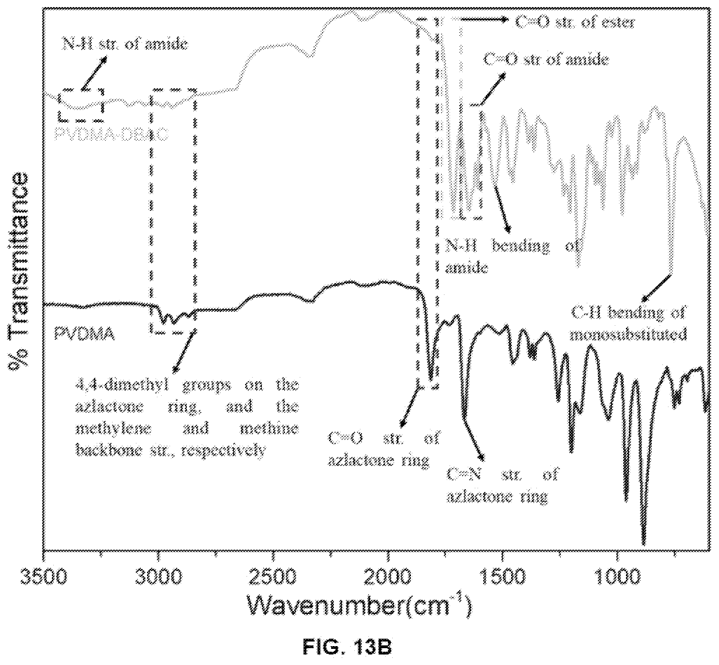

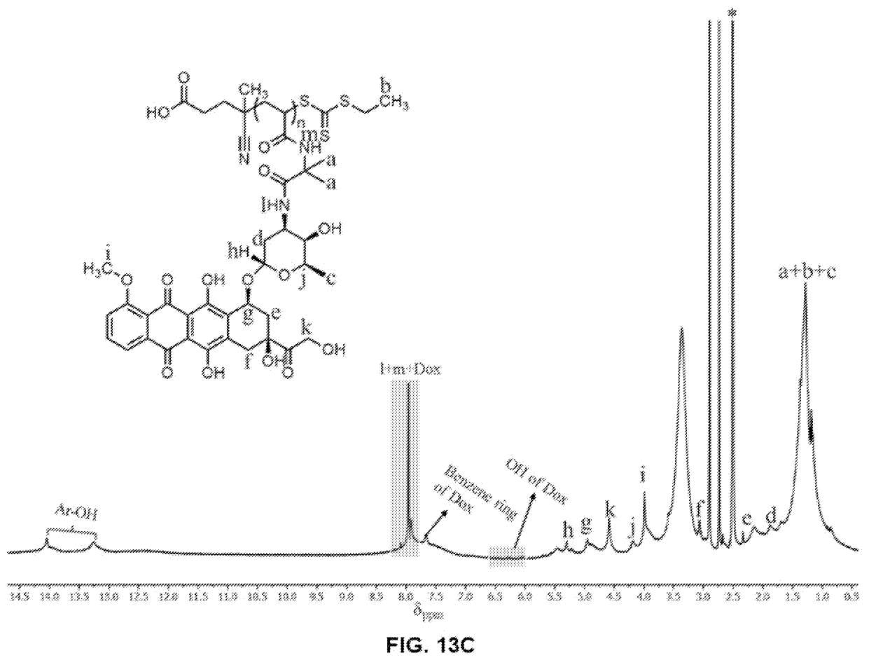

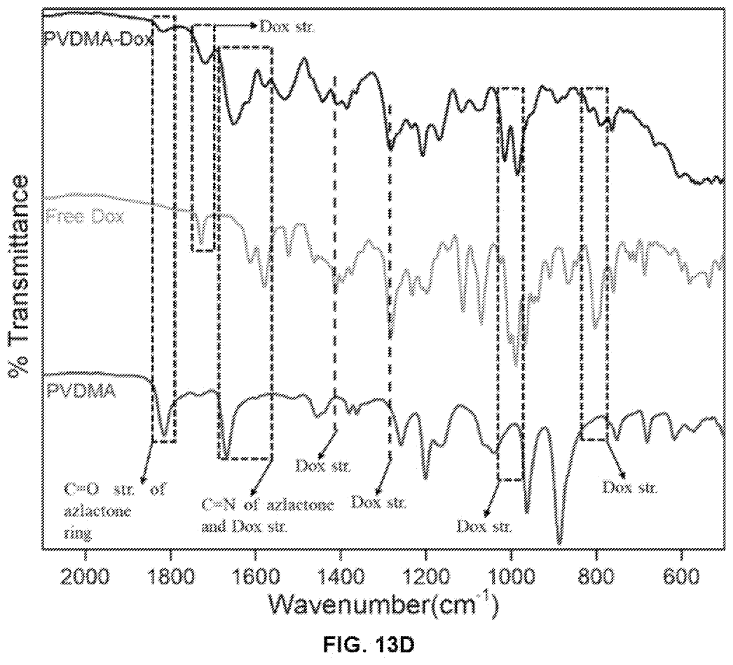

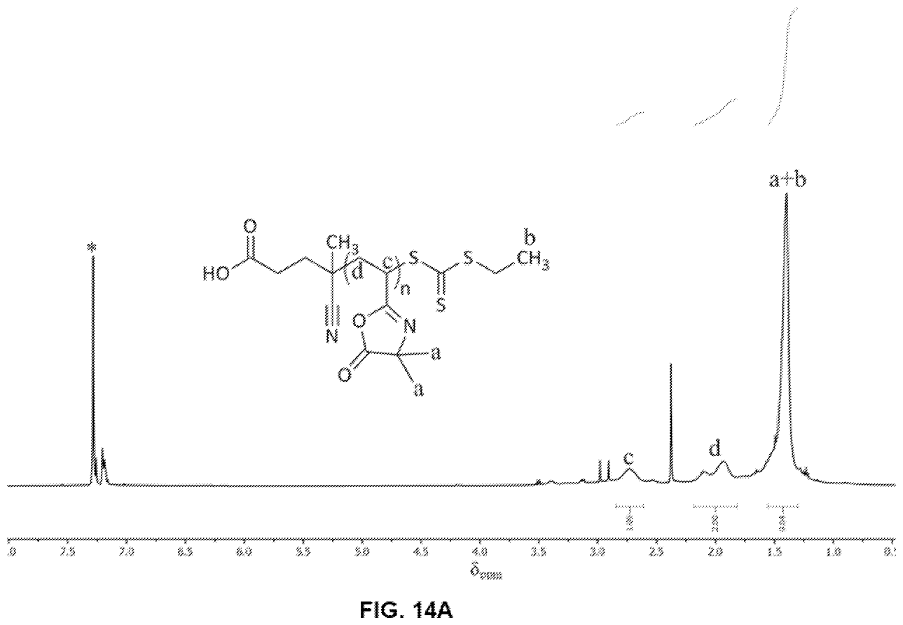

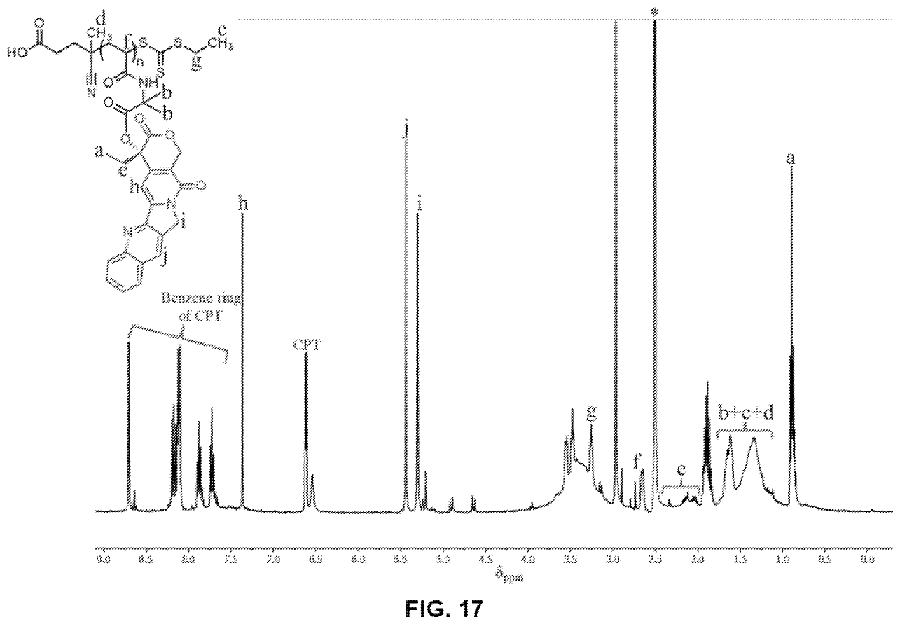

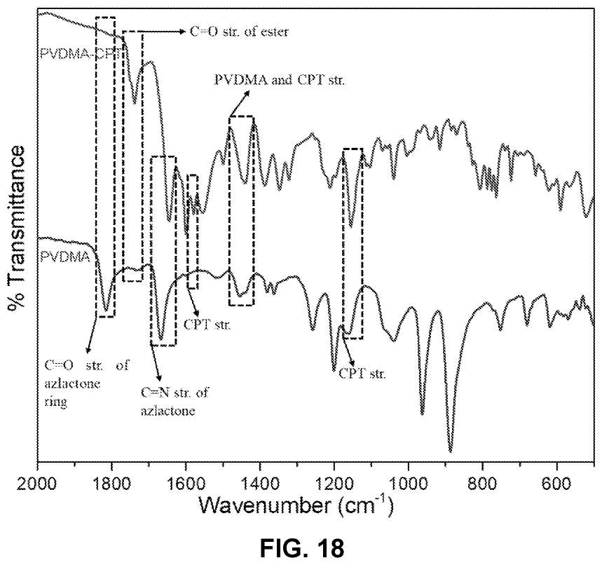

PVDMA (P3) was subjected to post-polymerization modification by DBAC (Scheme 1d), followed by ¹H-NMR and FT-IR spectroscopy to confirm the conjugation reaction. ¹H-NMR confirms the successful functionalization of the azlactone ring of PVDMA polymer by a DBAC (FIG. 13A). In ¹H NMR spectra peak at 2.46-2.12 ppm correspond to the polymer backbone, 3.59 and 3.27 ppm indicated presence of the DMAE associated methyl groups, while peaks at 7.47-7.34, 7.77-7.66, and 8.04-7.89 ppm revealed aromatic ring protons of coumarin. Utilizing FTIR (FIG. 13B), a disappearance of the peak at 1810 cm⁻¹ for the C═O stretching frequency of the azlactone ring and a sharp, new peak at 1714 cm⁻¹ for the C═O stretching frequency of ester moieties were observed, in addition to a peak at 2975 cm⁻¹ revealing the C—H stretching frequency of the methyl group of the azlactone ring indicating the complete opening of the azlactone ring of PVDMA and substitution by DBAC. To exemplify the versatile nature of PVDMA as a scaffold for post-polymerization modification by a range of compounds, PVDMA was also modified with the anti-cancer drugs doxorubicin and camptothecin (Scheme 1e-f). Doxorubicin-modified PVDMA was structurally characterized by ¹H NMR (FIG. 13C) and FTIR spectroscopy (FIG. 13D). 1H NMR spectroscopy of doxorubicin-modified PVDMA polymer revealed a number of doxorubicin-associated peaks in the final conjugate, including aromatic carbon protons, aromatic ring-associated hydroxyl protons, and others. FTIR spectroscopy exhibited a peak at 1720 cm⁻¹, indicating C═O stretching frequency of doxorubicin, and the peaks at 1652 and 1533 cm⁻¹ for C═O stretching and N—H bending of amide, respectively. ¹H NMR (FIG. 17) and FTIR spectroscopy (FIG. 18) also demonstrated the successful modification of the PVDMA scaffold by the camptothecin drug.

Functionalization of PVDMA with DMAE for Nucleic Acid “Catch and Release”

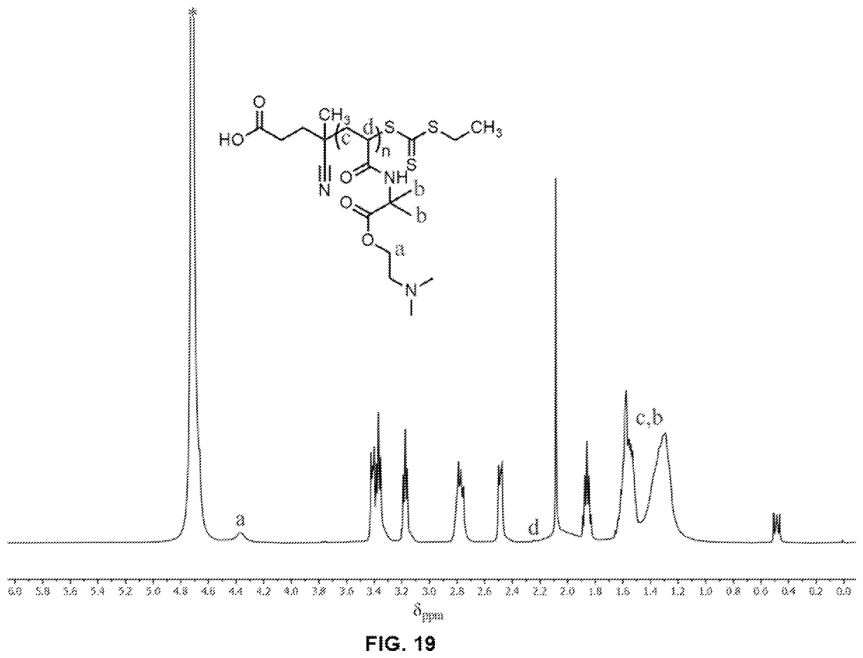

The versatile nature of PVDMA as a drug delivery platform was further shown by forming charge-reversing polymers that could be used to “catch and release” nucleic acids for improved intracellular delivery. To do so, PVDMA was modified with the tertiary amine-containing amino alcohol DMAE. The DMAE-modified PVDMA was characterized by ¹H-NMR spectroscopy and confirmed the presence of ester moieties (peak at 4.48-4.24 ppm corresponding to the O═C—O—CH₂— proton peak, FIG. 19). In the synthetic scheme, DBU helped to deprotonate DMAE by forming DBU·[DBU-H]⁺-DMF-H₂O or DBU·[DBU-H]⁺-DMF as previously reported. Further, the azlactone ring of PVDMA opened by reacting with the activated amino ethanol, leading to hydrolyzable ester linkages between PVDMA and DMAE side groups.

Polyplex Formation and Nucleic Acid Release from DMAE-Modified PDMA

The ability of DMAE-modified PVDMA to form polyplexes with nucleic acids, followed by subsequent hydrolysis and nucleic acid release, was assessed by agarose gel electrophoresis after complexation with a short, scrambled, double-stranded DNA sequence (FIG. 8A). As shown in FIG. 8B, DMAE-modified PVDMA can complex DNA at N/P ratios of 25, at which the DNA mobility through the gel is completely inhibited. Since DMAE-modified PVDMA has been reported to undergo hydrolysis and a cationic-to-anionic shift in charge near physiological pH values, the ability of DMAE-modified PVDMA to release nucleic acid under physiologic conditions was investigated. To examine the timed release of the scrambled DNA from the polyplexes via the hydrolysis of the ester linkages, polyplexes were formed at N/P ratios of 25 and then incubated at pH 7.4 and 37° C. for times ranging from 0-24 hours. As shown in FIG. 8C, DNA mobility was no longer retarded above 6 hours, indicating the successful hydrolysis of the ester linkages of DMAE-modified PVDMA and nucleic acid release.

Drug Release Kinetics of DBAC from DBAC-Modified PVDMA

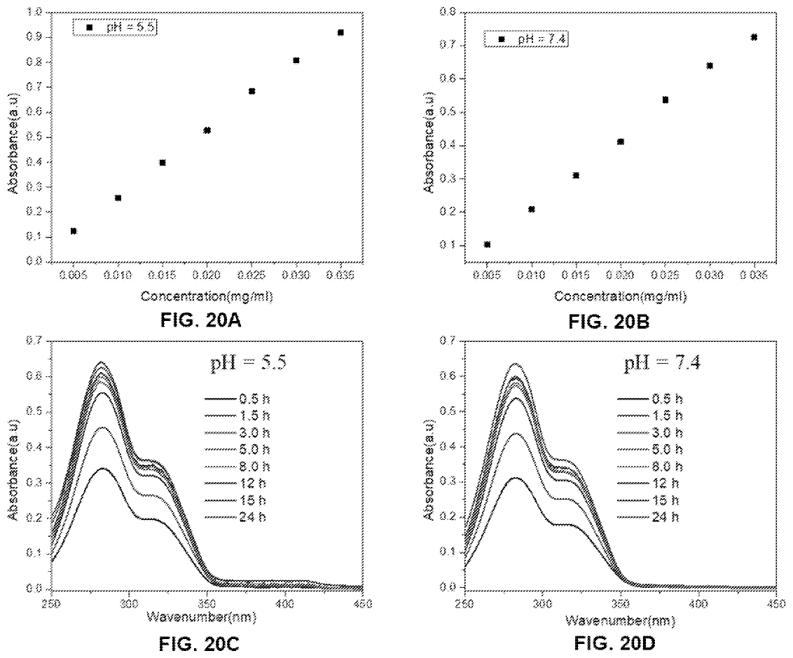

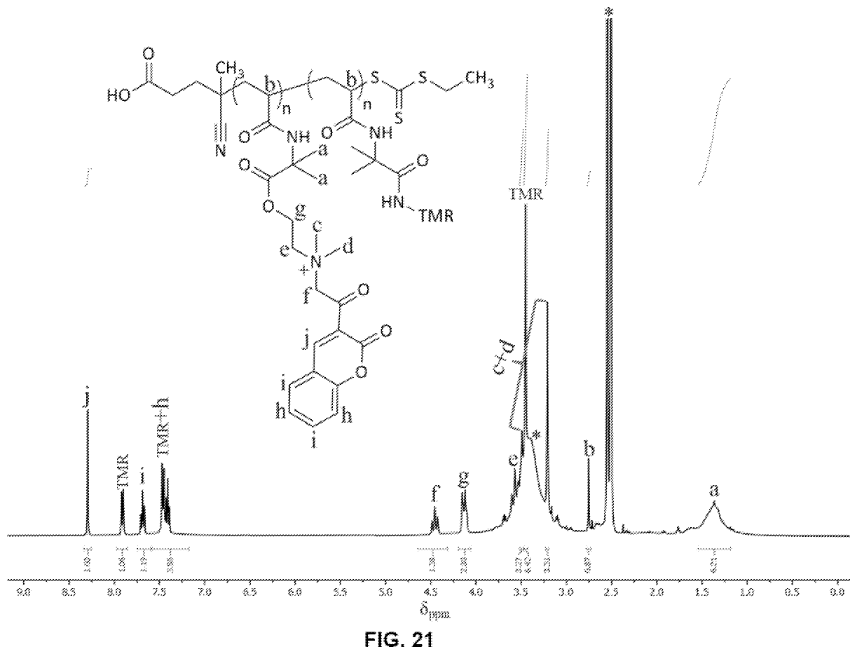

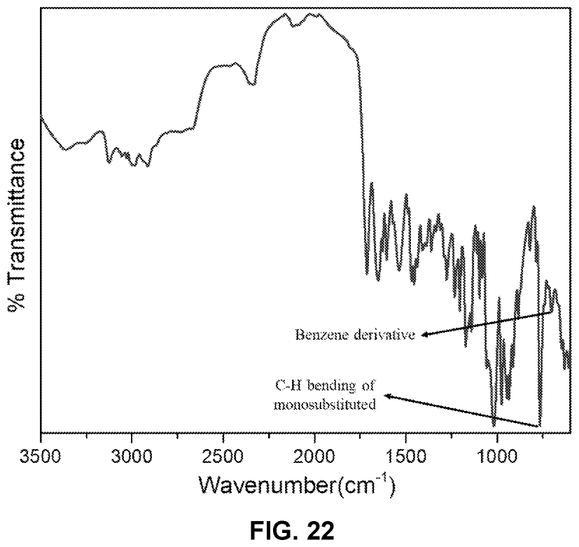

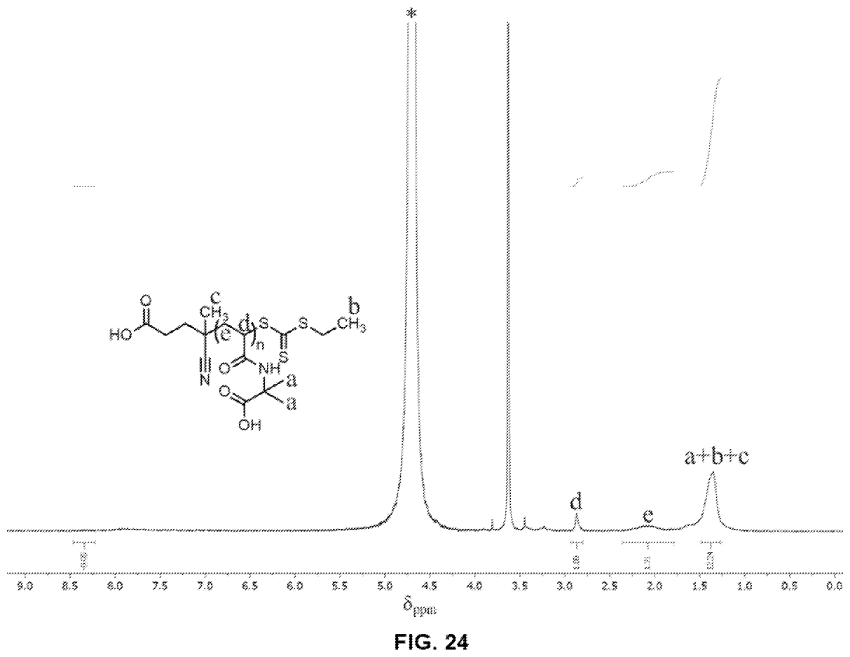

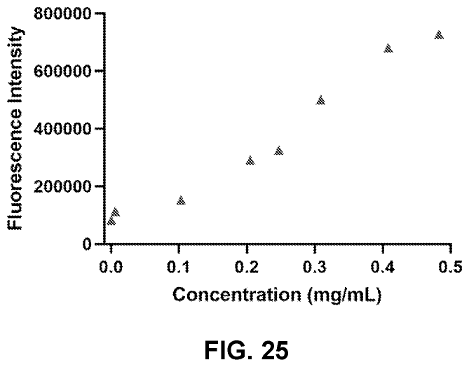

Similarly, it was desirable to show that the small molecule DBAC could be liberated from DBAC-modified PVDMA via hydrolysis of ester linkages between DBAC and PVDMA (FIG. 8D). In order to study the release of DBAC from PVDMA, DBAC-modified PVDMA was dialyzed against various pH solutions (5.5 and 7.4) at 37° C. and monitored the absorbance of DBAC (316 nm) in supernatants collected from dialysis baths via UV-Vis spectroscopy (FIGS. 20A-20D). At 0.5 h, the release of DBAC was observed to be 28% and 25% for pHs 7.4 and 5.5 respectively. The DBAC release rate was followed the similar trend up to 5 h. After 5 h, the rate of release of DBAC reached a plateau, while it was increases very slowly for both pH 5.5 and 7.4. In between pHs 5.5 and 7.4, the DBAC release rate was comparatively faster at pH 7.4, suggesting that the ester hydrolysis of DBAC-modified PVDMA (and subsequent release of DBAC) is accelerated at higher pH (FIG. 8E). ¹H NMR spectra were obtained of the DBAC-modified PVDMA before and after hydrolysis and are shown in FIGS. 23-24, respectively. These spectra show that the PVDMA polymer is 100% substituted by DBAC based on the ratio of aromatic protons at ˜8.3 ppm to azlactone methyl peaks at ˜1.3 ppm. Moreover, the disappearance of the coumarin aromatic moieties (8.3-8.0 ppm) from the polymer backbone after 24 h incubation in pH 7.4 buffer indicate complete hydrolysis of the ester linkage. The overall trend of decreasing hydrolysis rate is in agreement with previous observations of the hydrolysis rate of ester-containing polymers. For example, in their study of the hydrolytic stability of poly(2-(dimethylamino)ethyl methacrylate in water, Wetering attributed the decreasing hydrolysis rate to the formation of carboxylic acid groups which cause a decrease in the overall polymer charge and thus decrease the electrostatic attraction of hydroxyl ions. Since the hydrolysis of the ester moieties which link the coumarin to the polymer backbone lead to the formation of carboxylate moieties, it is believed that this explains the observed trend displayed in FIG. 8E.

### Biocompatibility of DBAC-Modified PVDMA

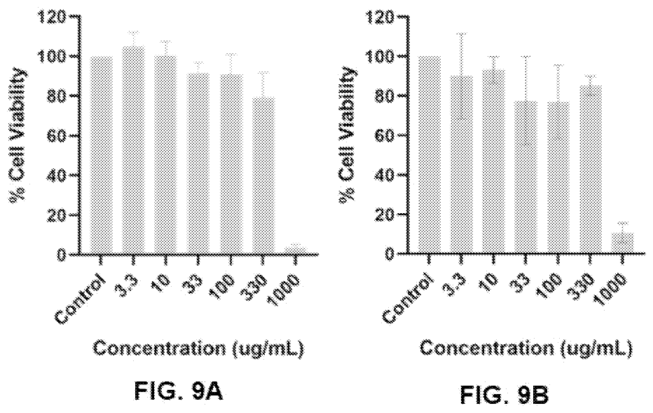

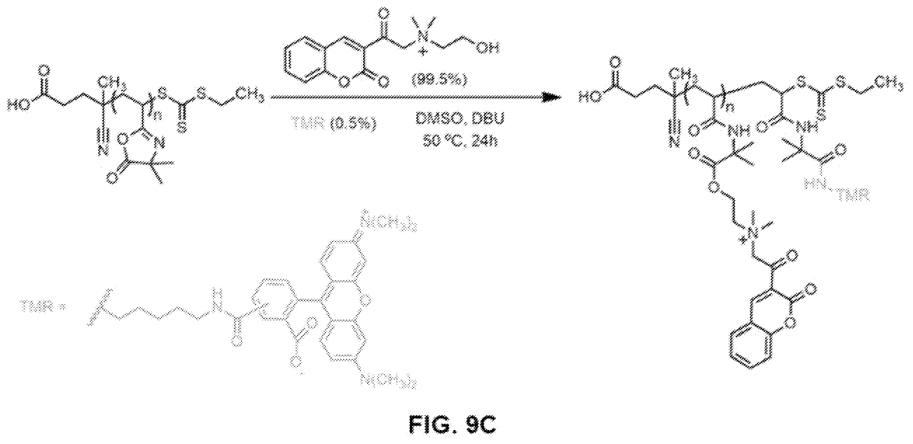

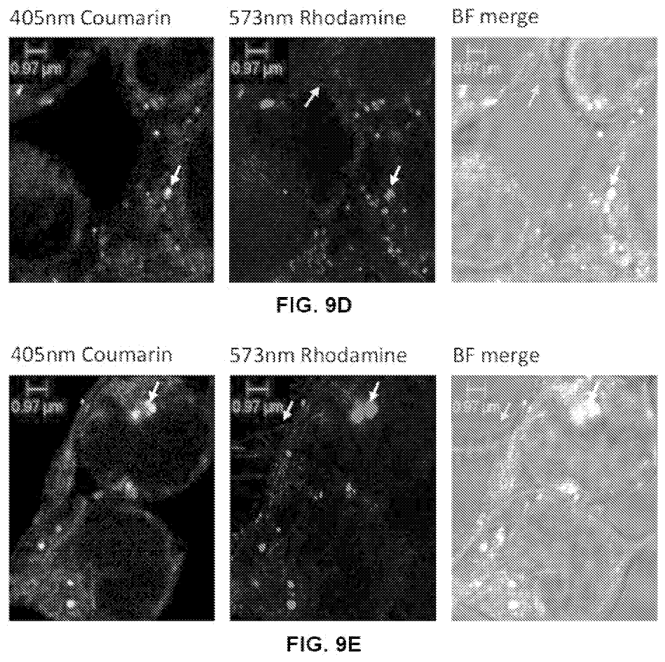

Having shown the versatility of PVDMA to form a variety of drug delivery constructs, the biocompatibility, cell trafficking, and biodistribution of PVDMA were demonstrated using DBAC-modified PVDMA as a representative polymer-drug conjugate. DBAC-modified PVDMA was chosen to move forward because of its promising physicochemical characteristics and the exciting potential of coumarin-based drugs in a variety of pathological settings. The cytotoxicity of DBAC-modified PVDMA was assessed by measuring the cell viability of multiple cell lines (HeLa and HEK-293) after incubation with the polymers. For concentrations of DBAC-modified PVDMA up to 330 μg/mL, HeLa and HEK-293 cell lines show viability above 75% after 24 h (FIGS. 9A-9B), indicating negligible acute cytotoxicity of the DBAC-modified PVDMA in cell culture.

Functionalization of PVDMA Polymer with Both DBAC and Tetramethyl Rhodamine Cadaverine (TMR)

To synthesize fluorescently-labeled DBAC-modified PVDMA polymer for cell trafficking studies, DBAC and TMR reagents were used to modify the azlactone ring of PVDMA at a stoichiometric ratio of 99.5:0.5 DBAC:TMR (FIG. 9C). 1H-NMR indicated new peaks arising at 3.44 ppm, 7.92-7.89 ppm, and 7.47-7.44 ppm when TMR is attached to the azlactone ring of PVDMA. Additionally, the C═O stretching frequency of the azlactone ring at 1810 cm⁻¹ completely disappeared after the reaction with DBAC and TMR. Moreover, a peak associated with the ring vibration of TMR appeared at 701 cm⁻¹, confirming the ring opening reaction of the PVDMA polymer with DBAC and TMR was successful.

### Uptake and Subcellular Distribution of DBAC-TMR-PVDMA Polymers

The intracellular trafficking of DBAC-TMR-PVDMA polymers was investigated using super-resolution confocal microscopy of HEK-293 cells, where PVDMA was monitored fluorescently by the irreversible attachment of TMR (i.e., rhodamine derivative) to the polymer backbone. Initially, a pulsed treatment approach was used, where cells were incubated with DBAC-TMR-PVDMA (PVDMA modified with 99.5% DBAC and 0.5% TMR) for 10 mins, followed by removal of treatment media and imaging. Clear and prominent accumulation of both DBAC and TMR within lysosomes after 10 mins of treatment was observed, indicating colocalization at these early time points (FIG. 9D). Moreover, minimal interaction of the DBAC-TMR-PVDMA polymers with cell or endolysosomal membranes was observed, suggesting that the polymers maintained water solubility in the cellular environment and were inert to the lipid bilayers of the cell. Next, the distribution of DBAC-TMR-PVDMA polymer within cells following overnight incubation was investigated (i.e., with prolonged exposure to the polymer). Again, an apparent accumulation of both DBAC and TMR was observed within the lysosomes of the cells (FIG. 9E). However, in the case of overnight incubation, clear cytosolic distribution of the DBAC was observed, as well. This cytosolic distribution was not apparent for TMR from the polymer, suggesting that DBAC was intracellularly liberated from the polymer backbone. Based on the early accumulation of DBAC and TMR within lysosomes of the cells, it was anticipated that cleavage of DBAC from the polymer backbone takes place within the lysosomal compartment, followed by diffusion of liberated DBAC into the cytosol. Finally, cells were pulsed with 10 mins of treatment followed by time-lapse imaging for 60 mins after treatment. Time-lapse images again revealed rapid accumulation of DBAC-TMR-PVDMA intracellularly with punctate staining that indicated lysosomal colocalization (FIG. 9F). Additionally, a considerable loss of signal was observed from 10 mins post-treatment to 60 mins post-treatment, which indicates degradation and/or release of the polymers from the cells over time.

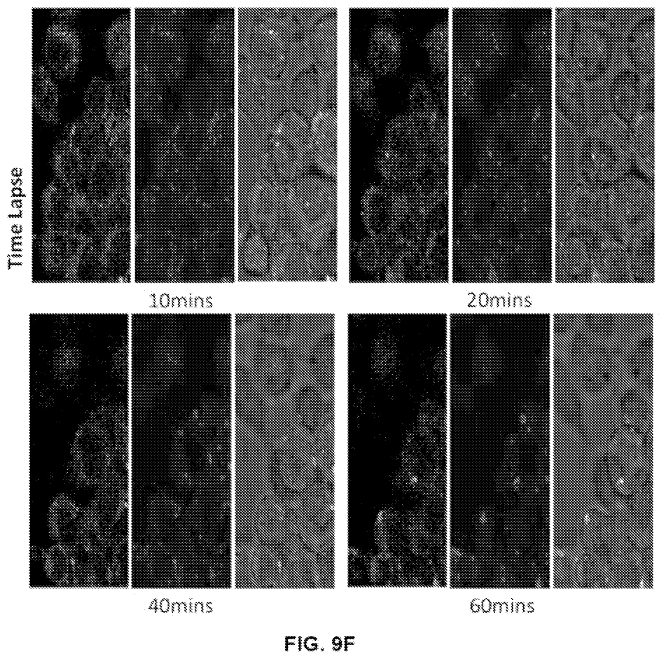

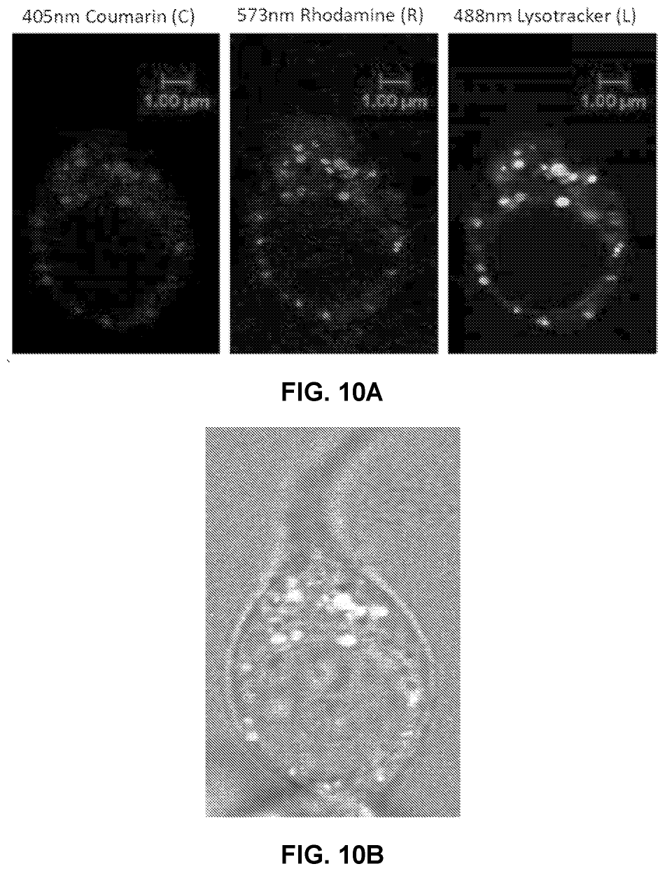

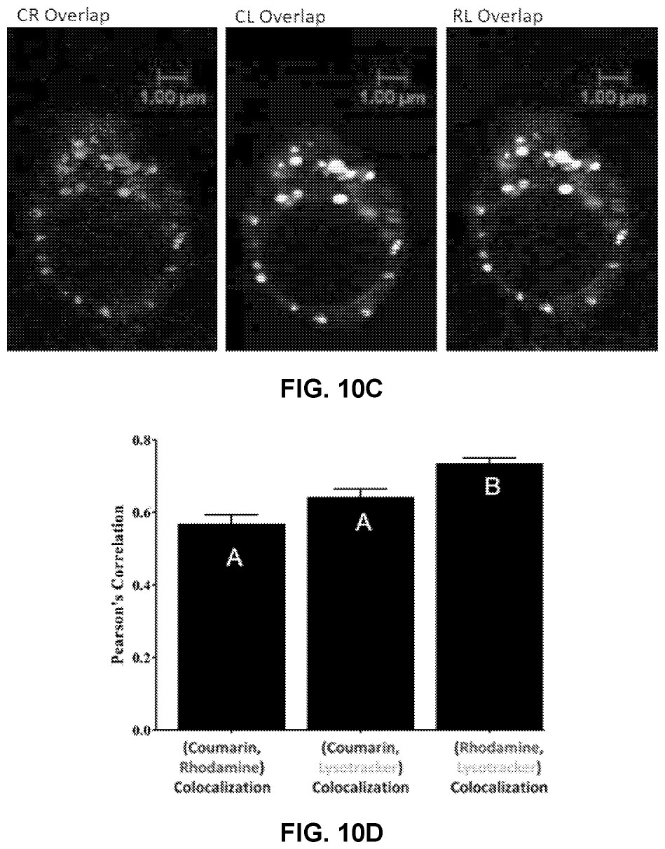

To investigate this disparate trafficking of DBAC and the PVDMA polymers (labeled with TMR) further, colocalization experiments were performed after overnight incubation with HEK-293 cells. In addition to monitoring DBAC and TMR fluorescence, lysosomes were also labeled using LysoTracker Green and quantified the colocalization of DBAC, TMR, and LysoTracker (FIGS. 10A-10C). As expected, extremely high colocalization of TMR was observed (i.e., polymer) and LysoTracker, with a Pearson's Correlation Coefficient of ˜0.8 (FIG. 10D). DBAC colocalized with lysosomes to a significantly lesser degree, with a 25% reduction in LysoTracker colocalization compared to TMR and Lysotracker. DBAC and TMR colocalization was further reduced, with a Pearson's Correlation Coefficient of less than 0.6. This further supports the notion that DBAC diffuses into the cytosol following its liberation from PVDMA within the lysosomes of the cell.

### Pharmacokinetics and Biodistribution of DBAC-TMR-PVDMA Polymers

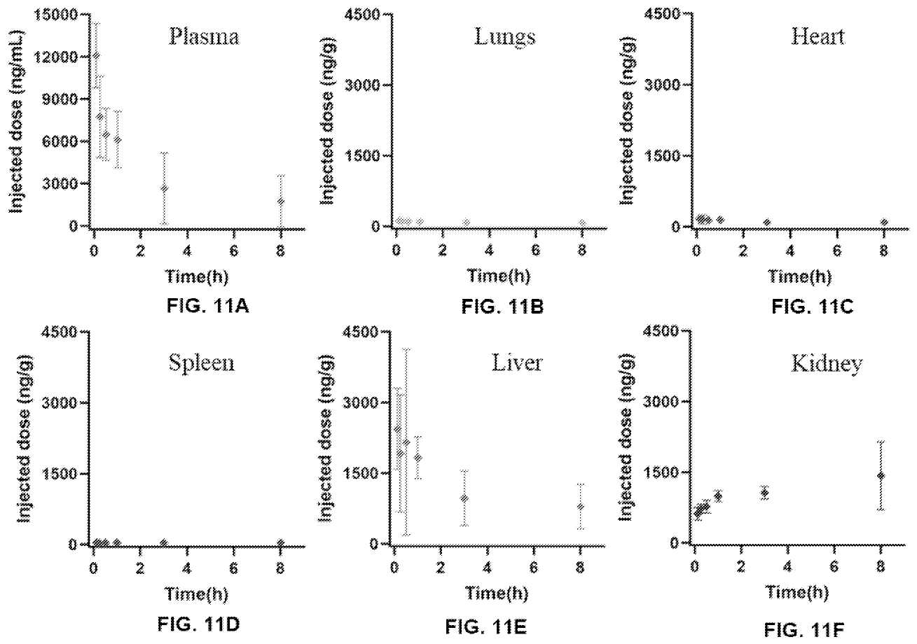

The pharmacokinetics and biodistribution of DBAC-TMR-modified PVDMA polymers were investigated, since PVDMA-based polymers have broad potential utility for drug delivery but have not been validated to date in vivo. Moreover, the delivery of DBAC-TMR-PVDMA (TMR was included for fluorescent quantification of polymer) was investigated by both the oral and intravenous delivery routes to establish the potential for delivery of DBAC-modified PVDMA polymer-drug conjugates via each of these major delivery routes. Upon IV administration at 10 mg/kg to CD-1 mice, DBAC-TMR-PVDMA polymer persisted well within the plasma with a circulation half-life (t_{1/2}) of 2.45±0.98 hrs and area under the curve (AUC) of 27,491±6563 ng·h/mL (FIG. 11A and Table 3). Interestingly, relatively low exposure of the DBAC-TMR-PVDMA polymer was seen within the lungs, spleen, and heart (AUCs of 599±105, 302±14.9, and 863±143 ng·h/g, respectively), while high exposure was observed in the liver (AUC=9,261±770 ng·h/g) (FIGS. 11B-11E and Table 3). High exposure of DBAC-TMR-PVDMA was also seen within the kidneys after IV administration (AUC=9,059±2274 ng·h/g) (FIG. 11F). Moreover, the concentration of DBAC-TMR-PVDMA in the kidneys steadily increased over time, suggesting that renal clearance was being observed and that DBAC-TMR-PVDMA was accumulating within the kidneys. Consequently, the time of maximum concentration (Tₘₐₓ) in the kidneys was substantially delayed compared to other compartments (˜8 hrs in kidneys as opposed to <1 hr in other compartments).

TABLE 3
Pharmacokinetic parameters of fluorescently-labeled polymer in CD1 mice following
intravenous and oral dosing of polymer-drug conjugate to mice at 10 mg/kg

| | Intravenous | | | | Oral | | |
|---|---|---|---|---|---|---|---|
| | PK parameters | | | | | | |
| | | Cₘₐₓ | AUC_{0-α} | | Cₘₐₓ | AUC_{0-α} | |
| | t_{1/2} | ng/mL or | ng × h/mL | F | ng/mL or | ng × h/mL | Tₘₐₓ |
| | h | ng/g | or ng × h/g | % | ng/g | or ng × h/g | h |
| Plasma | 2.45 ± 0.98 | 12080 ± 2280 | 27491 ± 6563 | 78.91 ± 20.28 | 6651 ± 1041 | 21693 ± 5570 | 2.00 ± 1.15 |
| GI tract | 4.27 ± 1.83 | 3807 ± 3122 | 6235 ± 3997 | 229 ± 38.84 | 1504 ± 378 | 9155 ± 1552 | 2.63 ± 2.50 |
| Heart | NA | 197 ± 69.8 | 863 ± 143 | 1013 ± 268 | 168 ± 33 | 1451 ± 384 | 1.25 ± 1.19 |
| Lung | NA | 113 ± 8.14 | 599 ± 105 | 1128 ± 334 | 256 ± 179 | 1186 ± 351 | 1.25 ± 1.19 |
| Spleen | NA | 51.7 ± 11.7 | 302 ± 14.9 | 1845.13 ± 420.67 | 27 ± 9 | 275 ± 63 | 8.00 ± 8.08 |
| Liver | NA | 3037 ± 967 | 9261 ± 770 | 956.77 ± 375.98 | 2082 ± 218 | 7363 ± 2893 | 1.25 ± 1.19 |
| Kidney | NA | 1440 ± 706 | 9059 ± 2274 | 85.7 ± 32.3 | 401 ± 178 | 1950 ± 735 | 1.75 ± 1.44 |

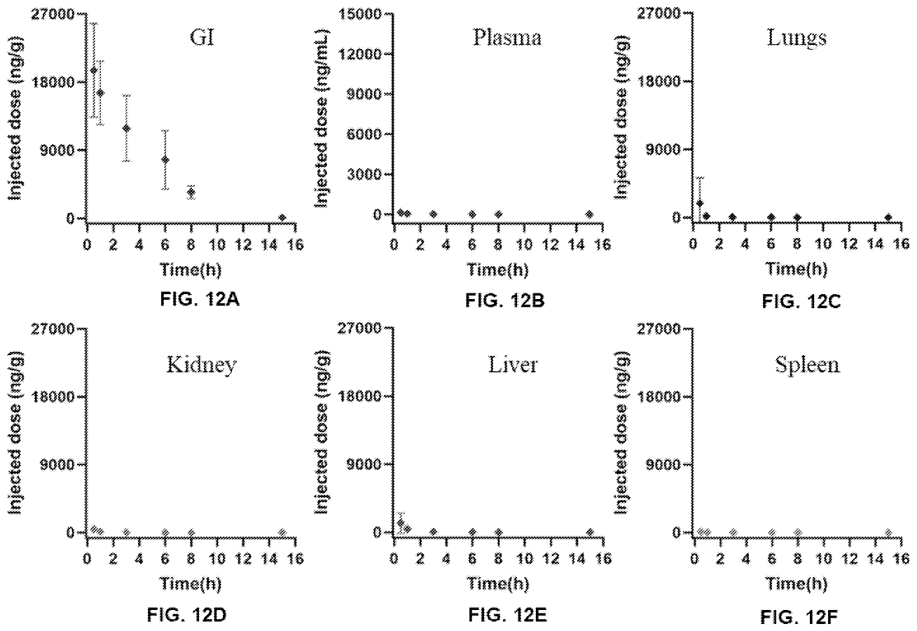

Next, the biodistribution of DBAC-TMR-PVDMA polymers after oral administration in CD-1 mice was investigated. Unsurprisingly, oral administration of DBAC-TMR-PVDMA led to very high exposure in the gastrointestinal (GI) tract (FIG. 12A). The terminal half-life (t_{1/2}) in the GI tract after ingestion of 10 mg/kg DBAC-TMR-PVDMA was 6.97±1.03 h. Moreover, the AUC of the drug in the GI tract was 95,643±1023 ng·h/g after oral administration, which was 5.15× greater than after IV administration (18,544±156 ng·h/g). Interestingly, minimal absorption of the DBAC-TMR-PVDMA polymers into the systemic circulation and other organs was found (FIGS. 12B-12F). Indeed, the AUC of DBAC-TMR-PVDMA in all other compartments other than the GI tract was substantially lower via the oral route as compared to IV administration, and negligible drug concentrations were measured in these compartments beyond 1 hr post-administration (Table 4).

TABLE 4
Pharmacokinetic parameters of DBAC generated from polymer-drug conjugate in CD1 mice following
intravenous and oral dosing of polymer-drug complex conjugate to mice at 10 mg/kg

| | Intravenous | | | | Oral | | |
|---|---|---|---|---|---|---|---|
| | PK parameters | | | | | | |
| | | C_{O} | AUC_{0-α} | | Cₘₐₓ | AUC_{0-α} | |
| | t_{1/2} | ng/mL or | ng × h/mL | F | ng/mL or | ng × h/mL | Tₘₐₓ |
| | h | ng/g | or ng × h/g | % | ng/g | or ng × h/g | h |
| Plasma | 1.09 ± 0.12 | 7216 ± 1023 | 1586 ± 125 | 7.59 ± 2.48 | 119 ± 25.3 | 120 ± 65.0 | 0.75 ± 0.29 |
| GI tract | 6.97 ± 1.03 | 4899 ± 1104 | 18544 ± 156 | >100 ± 47.5 | 21079 ± 1102 | 95643 ± 1023 | 1.25 ± 0.53 |
| Heart | 2.20 ± 0.78 | 4961 ± 715 | 8914 ± 512 | 11.3 ± 2.57 | 617 ± 85.6 | 1008 ± 95.6 | 4.38 ± 0.53 |
| Lung | 3.73 ± 0.47 | 1326 ± 202 | 1772 ± 141 | 74.2 ± 25.3 | 1955 ± 258 | 1315 ± 85.6 | 1.25 ± 0.68 |
| Spleen | 2.27 ± 0.55 | 1026 ± 248 | 1089 ± 124 | NA | NA | NA | NA |
| Liver | 2.24 ± 0.37 | 36898 ± 2501 | 17457 ± 3832 | 8.38 ± 1.98 | 1357 ± 253 | 1464 ± 346 | 0.63 ± 0.25 |
| Kidney | 3.49 ± 0.59 | 41296 ± 2103 | 10575 ± 1910 | 4.64 ± 2.59 | 477 ± 58.2 | 490 ± 101 | 0.50 ± 0.00 |

t_{1/2} = half life;
Cₘₐₓ/C₀: maximum concentration;
Tₘₐₓ: time at maximum concentration;
AUC_{0-α}: area under curve to infinite time;
F: relative bioavailability;
NA: not available

### Discussion

In this contribution, a template of PVDMA was developed as a versatile approach for drug conjugation and stimuli-responsive release via post-polymerization modification. Herein it is illustrated that a coumarin derivative, doxorubicin, or camptothecin could efficiently modify PVDMA scaffolds through post-polymerization modification. Moreover, it is demonstrated how charge-reversing delivery platforms can be created by altering PVDMA with a tertiary amine-containing alcohol (DMAE). Further, the nucleic acid release profile from polyplexes was shown herein, and it was found that DNA was effectively released from polyplexes after 6 hours. Additionally, it was shown that DBAC-modified PVDMA polymers are highly biocompatible and that DBAC is liberated from the polymer intracellularly for cytosolic accumulation. Finally, the PVDMA scaffolds were demonstrated to deliver DBAC drug in vivo by monitoring the pharmacokinetics and biodistribution of a PVDMA-based polymer for the first time and exhibited distinct accumulation profiles depending on the administration route (i.e., intravenous vs. oral).

A desirable approach to simplifying drug delivery for a variety of clinical purposes is drug conjugation to polymeric structures. The formation of polymer-drug conjugates has high utility for increasing drug solubility and bioavailability, improving drug pharmacokinetics, enabling targeted drug delivery, and producing combination therapies with precise ratiometric dosing. However, currently offered polymers for drug conjugation often have limited chemical flexibility for subsequent drug loading. PVDMA, on the other hand, offers a wide range of reactive flexibility and has been used for modification by a range of therapeutic compounds. In 2001, Heilmann et al. reported ring-opening modification of azlactones with alkyl and aryl nucleophiles, including primary amines, alcohols, and thiol functionalities. The Lynn group developed multilayered thin films using PVDMA-based ‘charge-shifting’ cationic polymers for DNA release or delivery from the thin films where the release of the DNA could be controlled by the layering scheme and by tuning the ratio of hydrolyzable side chains (e.g., esters) to non-hydrolyzable side chains (e.g., amides). Buck and Moore reported the conjugation of holo-transferrin and ovotransferrin proteins into the backbone of PVDMA scaffolds for applications in targeted drug delivery. To demonstrate the conjugation of this reactive polymer with proteins in an aqueous solution, they first functionalized PVDMA with triethylene glycol monomethyl ether (mTEG) to generate water-soluble PVDMA scaffolds, followed by attachment to the proteins of interest. Therefore, PVDMA could offer the ability to create a range of exciting polymer-drug conjugates with desirable properties such as broad chemical flexibility, optimizable structure/architecture, multivalent modifications, and highly tunable drug release.

Importantly, this is the first report utilizing PVDMA as a reactive scaffold to form polymer-drug conjugates. In this study, DBAC-modified PVDMA polymer was demonstrated to be readily soluble in water, likely due to the incorporation of the tertiary amine of DMAE in the final structure of the conjugate. Since the water solubility of PVDMA is low compared to other hydrophilic polymers utilized for polymer-drug conjugates in the past, this approach could improve the applicability of PVDMA for producing a range of polymer-drug conjugates. Moreover, this approach could be adapted to a range of other hydrophobic drugs in order to produce more hydrophilic PVDMA-based polymer-drug conjugates with improved bioavailability. Since PVDMA had never been assessed in vivo, the biodistribution of DBAC-TMR-PVDMA polymers was checked upon IV and oral administration in CD-1 mice. When administered through the IV route, DBAC-TMR-PVDMA persisted well within the plasma and had relatively low exposure in the lungs, spleen, and heart. While high exposure of DBAC-TMR-PVDMA in the liver and kidneys was observed, as expected, it was intriguing to see the steady accumulation and retention of DBAC-TMR-PVDMA in the kidneys over time. It is, therefore, exciting to speculate that PVDMA could be used for targeted drug delivery to the kidneys in future applications. However, more work is needed to confirm the exact location within the kidneys which is being targeted and to confirm that increased renal retention does not lead to any long-term toxicities.

Further, the biodistribution of DBAC-TMR-PVDMA polymers after oral administration in CD-1 mice was studied. Unsurprisingly, it was observed that the ingestion of DBAC-TMR-PVDMA led to very high exposure in the gastrointestinal (GI) tract compared to other organs. However, it was interesting to see that systemic exposure of DBAC-TMR-PVDMA remained very low over time, suggesting that DBAC-TMR-PVDMA polymers could remain sequestered within the GI tract and prevent systemic absorption of the drug conjugated to PVDMA. This could be a promising approach to achieve highly-selective GI delivery of compounds that cause toxicities when absorbed systemically. Moreover, this approach pairs well with the base-catalyzed nature of drug release from PVDMA since slightly elevated pHs in the GI tract will further enable targeted drug release at this site.

In addition to showing the utility of PVDMA as a generic polymer template for drug modification, DBAC-modified PVDMAs could also have strong therapeutic potential. Many coumarin derivatives (similar to the BAC used in these studies) have desirable pharmacological properties, including antiviral, antiproliferative, antioxidant, anticoagulant, anti-HIV, and anti-cancer activities. These coumarins have a straightforward structure, and because of their versatility, they are used for a wide range of applications, including in the cosmetic, pharmaceutical, food, and fragrance industries. However, many derivatives in this class of molecules with wide-ranging physicochemical properties have limited utility because of their poor water solubility and bioavailability. Moreover, there are reports of hepatoxicity associated with some of the coumarins, which could be counteracted particularly by oral administration of the DBAC-modified PVDMAs introduced here due to their low systemic absorption and efficient GI targeting. Coumarin derivatives were conjugated with PVDMA as a novel polymer-drug conjugate synthesis technique to overcome these issues. The resulting DBAC-modified PVDMA constructs developed and validated here in cell culture and pharmacokinetic assays have a high potential for application to inflammatory diseases, cancers, and other pathophysiologies.

### CONCLUSION

In summary, poly(2-vinyl-4,4-dimethyl azlactone) is an efficient reactive tool for post-polymerization modification with various small molecule drugs and biologics. The versatile nature of PVDMA scaffolds have been demonstrated by modifying them with a coumarin-derivative (DBAC), doxorubicin, camptothecin, and DMAE (for nucleic acid complexation and release). In addition, controlled DBAC drug release from the PVDMA backbone was shown. Moreover, the modification of PVDMA scaffolds with tertiary amine-containing alcohols for making charge-reversing nucleic acid delivery platforms and demonstrate the controlled DNA release from polyplexes have been demonstrated herein. The intracellular trafficking of a PVDMA derivative has been shown for the first time and observed that DBACs are liberated from the polymer intracellularly for cytosolic accumulation. Importantly, the pharmacokinetics and biodistribution of a PVDMA-based polymer were investigated for the first time and showed distinct accumulation patterns depending on the route of administration (i.e., IV vs. oral). The IV and oral routes of administration of DBAC-modified PVDMA could be useful for targeted drug delivery to the kidneys and GI tract, respectively. This work opens the door to using PVDMA as a versatile drug delivery template with wide-ranging applications, including the targeted delivery and controlled release of small molecule drugs, nucleic acids, and other biologics.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1. Aksakal, S. et al, Polym. Chem. 2018, 9, 4507-4516.
2. Bernhard, Y et al, Biomacromolecules 21(8) (2020) 3207-3215.
3. Boaen, N K et al, Chem. Soc. Rev. 34(3) (2005) 267-275.
4. Boisde, P M et al., In Kirk-Othmer Encyclopedia of Chemical Technology, John Wiley & Sons, Inc (Ed.)., 4 (2014) 1-10.
5. Buck, M. E. et al., ACS Appl. Mater. Interfaces 2010, 2, 1421-1429.
6. Buck, M. E. et al, Chem. Mater. 2010, 22, 6319-6327.
7. Buck, M. E. et al, Polym. Chem. 2012, 3, 66-80.
8. Carter, M. C. D. et al, Chem. Mater. 2016, 28, 5063-5072.
9. Carter, M. C. D. et al, Chem. Mater. 2020, 32, 6935-6946.
10. Chen, J. et al, J. Am. Chem. Soc. 2013, 135, 10938-10941.
11. Cotanda, P et al, J. Polym. Sci. Part A: Polym. Chem. 51(16) (2013) 3333-3338.
12. Cullen, S. P. et al, Langmuir 2008, 24, 13701-13709.
13. Davis, M E et al, Nat. Rev. Drug Discov. (2010) 239-250.
14. Deshmukh, S et al, Ferroelectrics 519(1) (2017) 23-32.
15. Donohue, J. J. et al., Langmuir 1989, 5, 671-678.
16. E. F. S. Authority, Coumarin in flavourings and other food ingredients with flavouring properties-Scientific Opinion of the Panel on Food Additives, Flavourings, Processing Aids and Materials in Contact with Food (AFC), EFSA J 6(10) (2008) 793.
17. Feng, J et al, Prog. Polym. Sci. 37(2) (2012) 211-236.
18. Fortenberry, A. et al, Macromol. Rapid Commun., 2022, 43, 2200420.
19. Gauthier, M A et al, Angew. Chem. Int. Ed. 48(1) (2009) 48-58.
20. Hansen, R. R. et al, Biomacromolecules 2013, 14, 3742-3748.
21. Heilmann, S M et al, J Polym Sci Part A. 39(21) (2001) 3655-3677.
22. Heilmann, S M et al, J. Polym. Sci. Part A: Polym. Chem. 39(21) (2001) 3655-3677.
23. Hong, J. et al, Biomacromolecules 2011, 12, 2975-2981.
24. Huang, J. et al, Chem. Soc. Rev. 42(17) (2013) 7373-7390.
25. Jérôme, C et al, Adv. Drug Delivery Rev. 60(9) (2008) 1056-1076.
26. Jewell, C M et al, Biomacromolecules 7(9) (2006) 2483-2491.
27. Kim, J. S. et al, Bioconjug. Chem. 2019, 30, 1220-1231.
28. Kumar, R. et al, Chem. Rev. 2021, 121, 11527-11652.
29. Lee, C C et al, Nat. Biotechnol. 23(12) (2005) 1517-1526.
30. Lee, Y. et al, Angew. Chemie-Int. Ed. 2009, 48, 5309-5312.
31. Levere, M. E. et al, Polym. Chem. 2011, 2, 2878-2887.
32. Liebscher, J. et al, Macromol. Chem. Phys. 2020, 221, 1900500.
33. Liu, J et al, Adv. Healthc. Mater. 5(15) (2016) 1868-1873.
34. Liu, J et al, Angew. Chem. Int. Ed. 127(3) (2015) 1016-1020.
35. Liu, X. et al, Biomacromolecules 2008, 9, 2063-2071.
36. Liu, X. et al, Macromolecules 2005, 38, 7907-7914.
37. Liu, X. et al, Soft Matter 2008, 4, 1688-1695.
38. Lokitz, B. S. et al, Macromolecules 2009, 42, 9018-9026.
39. Lokitz, B. S. et al, Macromolecules 2012, 45, 6438-6449.
40. Luten, J. et al, Bioconjug. Chem. 2006, 17, 1077-1084.
41. Luten, J. et al, J. Control. Release 2008, 126, 97-110.
42. Matos, M J et al, IntechOpen 25 (2015) 533-538.
43. Melagraki, G et al, Eur. J. Med. Chem. 44(7) (2009) 3020-3026.
44. Messman, J. M. et al, Macromolecules 2009, 42, 3933-3941.
45. Mohanty, A. K. et al, Macromolecules 2018, 51, 5313-5322.
46. Ooi, Y. J. et al, Biomacromolecules 2020, 21, 1136-1148.
47. Pedone, E. et al, J Mater. Chem. 13(11) (2003) 2825-2837.
48. Peer, D et al, Nat. Nanotechnol. (2020) 61-91.
49. Peng, X M et al, Curr Pharm Des 19(21) (2013) 3884-3930.
50. Quek, J. Y. et al, Macromolecules 2013, 46, 7290-7302.
51. Ristić, M N et al, Chem Biodivers 16(1) (2019) e1800486.
52. Ros, S. et al, ACS Omega 2020, 5, 9114-9122.
53. Ros, S. et al, Macromolecules 2018, 51, 5752-5761.
54. Ros, S. et al, Macromolecules 2020, 53, 3514-3523.
55. Sajan, D et al, Spectrochim. Acta A Mol. Biomol. Spectrosc. 82(1) (2011) 118-125.
56. Sandhya, B et al, Eur. J. Med. Chem. 46(9) (2011) 4696-4701.
57. Sinclair, A. et al, Biomacromolecules 2013, 14, 1587-1593.
58. Stiefel, C et al, ACS omega 2(8) (2017) 5242-5250.
59. Sun, B et al, Chem. Commun. 46(12) (2010) 2016-2018.
60. Sun, B. et al, J. Control. Release 2010, 148, 91-100.
61. Van de Wetering, P et al, Macromolecules 31(23) (1998) 8063-8068.
62. Veber, D F et al, J Med Chem. 45(12) (2002) 2615-2623.
63. Venugopala, K N et al, Biomed Res. Int. 2013 (2013).
64. Wang, G. et al, ACS Appl. Mater. Interfaces 2020, 12, 14825-14838.
65. Wong, S Y et al, Mol. Ther. 17(3) (2009) 480-490.
66. Zayas-Gonzalez, Y M et al, Biomacromolecules 17(9) (2016) 3067-3075.
67. Zhang, J. et al, Adv. Mater. 2007, 19, 4218-4223.
68. Zhang, J. et al, Langmuir 20(19) (2004) 8015-8021.
69. Zhang, M. et al, J. Control. Release 2020, 319, 46-62.
70. Zhang, S et al, J. Am. Chem. Soc. 134(44) (2012) 18467-18474.
71. Zhu, L et al, Org. Chem. Front. 6(15) (2019) 2678-2686.
72. Zhu, Y. et al, Macromolecules 2013, 46, 6475-6484.

1. A charge-shifting polymer comprising:
a. a polymeric backbone;
b. one or more tertiary amine-containing pendant groups; and
c. at least one linker between the polymeric backbone and each of the one or more pendant groups;
wherein the at least one linker comprises a hydrolyzable group.

2. The charge-shifting polymer of claim 1, wherein the hydrolyzable group comprises an amide, an ester, a thioester, or any combination thereof.

3. The charge-shifting polymer of claim 1, wherein the charge-shifting polymer has a number-average molecular weight (Mₙ) of from about 10,000 Da to about 24,000 Da.

4.-6. (canceled)

7. The charge-shifting polymer of claim 1, wherein the hydrolyzable group has a hydrolytic half-life of from about 15 hours to about 1800 hours at a temperature of from about 25° C. to about 50° C. and a pH of from about 5.5 to about 8.5.

8. (canceled)

9. A composition comprising the charge-shifting polymer of claim 1 conjugated to at least one therapeutic molecule.

10. The composition of claim 9, wherein the at least one therapeutic molecule comprises a small molecule, a peptide, a protein, DNA, RNA, or any combination thereof.

11. The composition of claim 10, wherein the small molecule comprises coumarin, a coumarin derivative, camptothecin, doxorubicin, tetramethylrhodamine cadaverine, or any combination thereof.

12. The composition of claim 11, wherein the coumarin derivative comprises DMAE-modified 3-bromoacetyl coumarin (DBAC).

13. The composition of claim 12, wherein the at least one therapeutic molecule comprises two small molecules.

14. The composition of claim 13, wherein the two small molecules comprise DBAC and tetramethylrhodamine cadaverine.

15. (canceled)

16. The composition of claim 9, wherein the hydrolyzable group comprises at least a first hydrolyzable group and a second hydrolyzable group, and wherein the first hydrolyzable group and the second hydrolyzable group have different hydrolytic half-lives under a single set of pH and temperature conditions.

17. The composition of claim 16, wherein the first hydrolyzable group has a hydrolytic half-life that is shorter than a half-life of the second hydrolyzable group.

18. The composition of claim 16, wherein the hydrolyzable group further comprises a third hydrolyzable group having a different hydrolytic half-life from both the first hydrolyzable group and the second hydrolyzable group under a single set of pH and temperature conditions.

19. A method for controlled delivery of at least one therapeutic molecule to a subject, the method comprising administering the composition of claim 9 to the subject.

20. The method of claim 19, wherein hydrolysis of the charge-shifting polymer causes the at least one therapeutic molecule to disassociate from the charge-shifting polymer.

21. The method of claim 19, wherein hydrolysis of the charge-shifting polymer occurs intracellularly.

22. The method of claim 21, wherein hydrolysis of the charge-shifting polymer occurs in lysosomes.

23. The method of claim 20, wherein the hydrolyzable group comprises a thioester and the at least one therapeutic molecule dissociates from the charge-shifting polymer after uptake by a cell, wherein the cell comprises free glutathione.

24.-25. (canceled)

26. The method of claim 19, wherein the composition is administered orally and wherein the charge-shifting polymer has a terminal half life of from about 5 to about 9 hours.

27. The method of claim 19, wherein the subject is a mammal.

28.-35. (canceled)
